(19) United States
(12) Patent Application Publication
(10) Pub. No.: US 2026/0191759 A1
(43) Pub. Date: Jul. 9, 2026

(54) COSMETIC COMPOSITIONS AND METHOD FOR STABILIZATION AND CONTROLLABLE DELIVERY OF SKIN CARE ACTIVE COMPOUNDS

(71) Applicant: Momentive Performance Materials Inc., Niskayuna, NY (US)

(72) Inventors: Alok SARKAR, Electronic City (IN); Ashitha KANDIKKAL, Sarjapur (IN); Xu QIN, Northvale, NJ (US); Sarah Peeler BLACK, New York, NY (US); Shruthi ACHARYA, Dakshina Kannada (IN); George KURIAKOSE, Whitefield (IN); Benjamin FALK, Yorktown Heights, NY (US)

(21) Appl. No.: 19/427,710

(22) Filed: Dec. 19, 2025

(30) Foreign Application Priority Data
Dec. 20, 2024 (IN) 202411101416

(51) Int. Cl. A61K 8/85; A61K 8/41; A61K 8/49; A61K 8/67; A61K 8/9789; A61Q 19/00

(52) U.S. Cl. CPC A61K 8/85; A61K 8/41; A61K 8/498; A61K 8/676; A61K 8/9789; A61Q 19/00

(57) ABSTRACT

The invention relates to compositions, comprising at least one skin care active compound (A), and at least one crosslinked polyester component (B), comprising a crosslinked polyester and at least one cosmetically acceptable carrier (C), and optionally one or more other components commonly used in the cosmetic field (D), the components (A), (B), (C), and (D) each being different from each other. The compositions are, in particular, for the topical application to the skin or mucous membranes. They include, in particular, cosmetic skin care compositions. The invention further relates to the use of a crosslinked polyester component (B) for controlling or modifying the release of a skin care active compound (A) and for stabilizing a skin care active compound (A) in the cosmetic compositions.

The invention relates to compositions, comprising at least one skin care active compound (A), and at least one crosslinked polyester component (B), comprising a crosslinked polyester and at least one cosmetically acceptable carrier (C), and optionally one or more other components commonly used in the cosmetic field (D), the components (A), (B), (C), and (D) each being different from each other. The compositions are in particular for the topical application to the skin or mucous membranes. They include in particular cosmetic skin care compositions. The invention further relates to the use of a crosslinked polyester component (B) for controlling or modifying the release of a skin care active compound (A) and for stabilizing a skin care active compound (A) in the cosmetic compositions.

### Statement of the Problem

Active delivery through the skin represents a convenient route due to its non-invasive nature, avoiding pain, and the generation of medical waste. New materials are being developed to help avoid problems related to this versatile route, such as the degradation of active ingredients and uncontrolled release. Cosmetic industry in particular, has searched for many years for ways to improve the efficacy of active ingredients without compromising the key performance attributes of the cosmetic products such as skin-feel, moisturization and sensory. Consumer acceptance of cosmetic products is determined not only by their performance attributes but also how effectively they are able to stabilize the active ingredient therein during storage and deliver post-application. Active ingredients such as various forms of retinoids, ascorbic acid, tocopherols and niacinamide are useful in the treatment of diverse dermatological conditions, including inflammatory disorders, such as psoriasis, photoaging, age spots, skin wrinkles, acne, and skin cancers.

However, some actives in particular retinol, ascorbic acids are unstable and easily oxidized in presence of air as well as in the presence of ingredients commonly used in cosmetic formulation. The stabilization of these active become even more challenging when the product is applied as thin layer to a relatively large surface area like skin. These actives can also be very difficult to release depending upon the nature of cosmetic formulations, resulting in greatly reduced efficiency of delivery even with relatively high loading of active in the formulation. Accordingly, a formulation containing higher loading of an active can be less efficient compared to the formula containing lesser amount of active. Nonetheless, some of these actives for example retinols can also be irritating to the skin if their delivery is not controlled within a certain limit. Typically, retinol formulations having 4% to 6% of the active agent are used for dermatological applications, such as chemical skin peeling. Such formulations are recommended to be applied by professional staff or under medical supervision during skin treatments and are more prone to the aforementioned skin irritating affects (Rendon, M. et al “Evidence and considerations in the application of chemical peels. J. Clin. Aesthet. Dermatol. 2010, 3,32-43). Commonly used techniques that are designed for maintaining the stability and efficacy of sensitive active ingredients in cosmetic products includes emulsions, micro/nanoparticles and lipid-based nanocarriers. However, none of these technologies offer performance attributes like skin feel, moisturization and sensory.

The present invention relates to polyester elastomer compositions that helps maintain product stability upon storage and help to provide controlled release of active therein while improving the sensory and natural outlook of the skin. Such formulations and delivery systems disclosed herein include use as gel and as topical cream for dermatological and cosmetic skincare procedures.

### STATE OF THE ART

In the cosmetics field, emulsions of water-in-oil (W/O) or oil-in-water (O/W) and to some extent purely oil-based anhydrous formulations are used as vehicles for active delivery in skincare due to the fact that the oil can spread easily at the surface of the skin and form a unfirm film on the skin. However, these oil-based formulations are often criticized for being too greasy and tacky and for having a fragile stability. In addition, when such skin care actives are present in the oil phase of an oil-in-water or water-in-oil emulsion, it is often difficult to formulate as the actives tend to phase separate from the oil phase when the composition is applied to the skin. Therefore, less of the active present in the composition is actually delivered into the skin and the efficacy of such skin care actives become limited.

Moreover, in the simple polymer-based active delivery systems, the active substances are uniformly dispersed within a polymer in a form of a blend. Such systems typically exhibit first-order release behavior where the active molecules are released relatively rapidly initially, and the release significantly decelerates overtime. A particularly challenging delivery mode is zero-order, in which the dose of the delivered active is independent of time or residual concentration in the delivery vehicle.

U.S. Pat. No. 11,739,194 discloses glycerol-silicone elastomers as active matrix with controllable release profile. But silicone elastomer being extremely hydrophobic in nature, have limited versatility in terms of compatibility with polar solvents or emollients such as hydrocarbon oils, plant-based oils, glycerin, and water.

Polyesters are a class of compounds that contain an ester functional group in their polymer chain. The ester group can be hydrolyzed when treated with certain biological catalysts or certain mixed cultures of microorganisms which renders a large number of polyesters biodegradable. There is a growing interest in recent years to design and develop biobased polyesters from renewable resources such as emollients, emulsifiers, film formers, or other functional ingredients for personal care applications. See for example, U.S. Pat. Nos. 8,414,906; 9,334,358; 6,540,987; and 7,820,758. However, no polyester elastomer or polyester elastomer gel has yet been reported that provides the stability to air sensitive active ingredients during storage and deliver to the skin in a controlled manner at the site of application.

### DETAILED DESCRIPTION OF THE INVENTION

The present invention provides a cosmetic composition, comprising:
(A) at least one active ingredient,
(B) at least one crosslinked polyester component, comprising a crosslinked polyester which is reaction product of:
   (i) at least one compound selected from a polycarboxylic acid, a polycarboxylic acid ester, and combinations thereof,
   (ii) at least one polyol, and
   (iii) optionally one or more monofunctional component selected from
      (a) a monofunctional carboxylic acid; and
      (b) a monofunctional alcohol,
(C) optionally at least one cosmetically acceptable carrier, and
(D) optionally one or more other components commonly used in the cosmetic field, different from components (A), (B) or (C).

In an embodiment the cosmetic composition comprises:
(C) at least one cosmetically acceptable carrier, and
(D) optionally one or more other components commonly used in the cosmetic field, which are each different from components (A), (B) or (C).

The components (A), (B), (C) and (D) are each different from each other.

As will be described in more detail below the crosslinked polyester component (B) (sometimes called polyester elastomer) can be used as dry powder or as a gel which is suitably obtained by swelling the crosslinked polyester e.g. under shear force in a solvent (such as a low molecular weight emollient) to form a uniform polyester gel or paste having a wide viscosity range. The gel is usually a semi-solid colloid formed by suspending the crosslinked polyester in the solvent. The solid particles of the crosslinked polyester in the gel are supposed to form a three-dimensional network that captures and holds the solvent. The crosslinked polyester gel is usually a stable, jelly-like substance that is soft and flexible. These crosslinked polyester components (B) in particular provided as gels in a solvent are expected to deliver superior performance benefits such as improved sensory, structuring, and rheological performance and improved compatibility to the cosmetic compositions comprising the skin care active compound (A) compared to other elastomers disclosed previously.

The crosslinked polyester component (B) (polyester elastomer) comprises suitably the reaction product of:
(i) at least one compound selected from a polycarboxylic acid, a poly-carboxylic acid ester, and combinations thereof,
(ii) at least one polyol, and
(iii) (a) at least one mono-carboxylic acid; and optionally (iii) (b) one or more a mono-alcohol.

In an aspect, the crosslinked polyester component (B) is a reaction product of: compound (i) selected from the group consisting of
(1) one or more poly-carboxylic acids of formula (I)

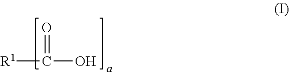

wherein
   R¹ is selected from the group consisting of C2-C52 alkyl group, C2-C52 heteroalkyl group, C2-C52 alkene group, C2-C52 heteroalkene group, C3-C52 cyclic group, or C2-C52 heterocyclic group, and
   a is an integer from 2 to 10, or
(2) one or more carboxylic acid esters of formula (II)

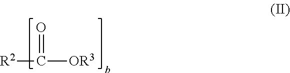

wherein
   R² is selected from the group consisting of C2-C200 alkyl group, C2-C200 heteroalkyl group, C2-C200 alkene group, C2-C200 heteroalkene group, C2-C200 alkyne group, C2-C200 heteroalkyne group, C2-C200 cyclic group, or C2-C200 heterocyclic group; and
   R³ is selected from the group consisting of C1-C22 alkyl group, C2-C22 alkylene group, or C3-C22 cyclic group, and
   b is an integer from 2 to 10, and
(3) a combination (1) and (2) thereof,
compound (ii) selected from one or more polyols of formula (III)

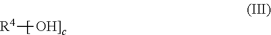

wherein
   R⁴ is C2-C200 alkyl group, C2-C200 heteroalkyl group, C2-C200 alkene group, C2-C200 heteroalkene group, C2-C200 alkyne group, C2-C200 heteroalkyne group, C3-C200 cyclic group, or C2-C200 heterocyclic group; and
   c is an integer from 2 to 10, and
   optional compound (iii) selected from the group consisting of
(a) one or more mono-carboxylic acids of formula (IV)

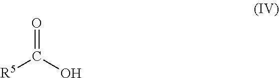

wherein
   R⁵ is C2-C52 alkyl group, C2-C52 heteroalkyl group, C2-C52 alkene group, C2-C52 heteroalkene group, C3-C52 cyclic group, or C2-C52 heterocyclic group, (b) one or more mono-alcohols of formula (V)

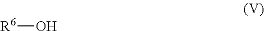

wherein
   R⁶ is C2-C52 alkyl group, C2-C52 heteroalkyl group, C2-C52 alkene group, C2-C52 heteroalkene group, C3-C52 cyclic group, or C2-C52 heterocyclic group.

In an aspect, the method of preparing a polyester elastomer (B) comprising reacting:
(i) at least one poly-carboxylic acid, at least one poly-carboxylic acid ester, or a combination thereof; and
(ii) at least one polyol; and/or
(iii) at least one mono-carboxylic acid; and/or
(iv) at least one mono-alcohol.

In an aspect, the crosslinked polyester elastomer (B) is the reaction product of:
(i) at least one polycarboxylic acid, at least one polycarboxylic acid ester, or a combination thereof; and
(ii) at least one polyol; and/or
(iii) at least one mono-carboxylic acid; and/or
(iv) at least one mono-alcohol.

In an aspect, the crosslinked polyester component (B) is reaction product of:
(i) one or more poly-carboxylic acids selected from di-carboxylic acids, tri-carboxylic acids and combinations thereof;
(ii) one or more polyols selected from diols, triols and combinations thereof, and
(iii) optionally one or more monofunctional carboxylic acids.

In an aspect, the crosslinked polyester (B) is reaction product of:
(i) one or more dicarboxylic acids
(ii) one or more triols, and
(iii) optionally one or more monofunctional carboxylic acids.

In an aspect, the preparation of crosslinked polyester component (B) is without solvent or emollient. In an aspect, the preparation of crosslinked polyester component (B) is with solvent or emollient as defined herein.

In an aspect, crosslinked polyester component (B) is only comprised of polyester. In an aspect, polyester elastomer is only comprised of crosslinked polyester. In an aspect, crosslinked polyester component (B) is comprised of crosslinked polyester and non-crosslinked polyester.

In an aspect, crosslinked polyester component (B) is comprised of polyester and solvent or emollient. In an aspect, crosslinked polyester component (B) is comprised of crosslinked polyester and solvent or emollient. In an aspect, the crosslinked polyester component (B) is comprised of crosslinked polyester, non-crosslinked polyester, and solvent or emollient.

In an aspect, the crosslinked polyester component (B) is a powder.

The crosslinked polyester component (B) form crosslinked polymer networks. As is well-known to a skilled person in the art such crosslinked polymer are not (completely) soluble and certain methods are available to characterize them, including for example sol-gel analysis determination of the gel fraction), swelling ratio analysis (determination of the swelling ratio), and mechanical analysis (e.g. determination of the modulus) (see e.g. Polym. Chem., 2024, 15, 219-247).

In an aspect, the fraction of the crosslinked polyester component (B) which is not soluble in ethyl acetate (gel fraction) is greater than or equal to 20%. In an aspect, the fraction of polyester elastomer which is not soluble in ethyl acetate (gel fraction) is greater than or equal to 40%. In an aspect, the fraction of the polyester elastomer which is not soluble in ethyl acetate (gel fraction) is greater than or equal to 50%. In an aspect, the fraction of the polyester elastomer which is not soluble in ethyl acetate (gel fraction) is greater than or equal to 60%. In an aspect, the fraction of the polyester elastomer which is not soluble in ethyl acetate (gel fraction) is greater than or equal to 70%. The gel fraction is suitably defined as

$$Gel fraction ( \% ) = 100 \times \frac{weight of dried gel ( insoluble residue of the extraction )}{total weiht of the polyester elastomer used in the extraction}$$

In an aspect, the gel fraction can be determined with an extraction method such as the Soxhlet extraction described herein.

In an aspect, crosslinked polyester component (B) is comprised of polyester elastomer and solvent or emollient as defined herein. In an aspect, polyester elastomer composition is comprised of polyester elastomer without solvent or emollient as defined herein. In an aspect, crosslinked polyester component (B) is a gel or a powder.

In an aspect, the crosslinked polyester component (B) is a polyester elastomer combined with one or more solvents or emollients, which can be converted to a polyester elastomer gel (the swollen polyester elastomer), for example, by applying a shear force to the composition.

In an aspect, the present disclosure provides for the use of a gel, or a powder prepared from a polyester elastomer described herein, in the manufacture of coloured cosmetic or personal care formulations.

### DETAILED DESCRIPTION

### I Definitions

Unless otherwise indicated, any atom with unsatisfied valences is assumed to have hydrogen atoms sufficient to satisfy the valences.

It is to be noted that the term “a” or “an” entity refers to one or more of that entity; for example, “a nucleic acid sequence,” is understood to represent one or more nucleic acid sequences, unless stated otherwise. As such, the terms “a” (or “an”), “one or more,” and “at least one” can be used interchangeably herein.

Furthermore, “and/or” where used herein, is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

As used herein, the following definitions shall apply unless otherwise indicated. For purposes of the present disclosure, the chemical elements are identified in accordance with the Periodic Table of the Elements, CAS version, and the Handbook of Chemistry and Physics, 75th Ed. 1994. Additionally, general principles of organic chemistry are described in “Organic Chemistry,” Thomas Sorrell, University Science Books, Sausalito: 1999, and “March's Advanced Organic Chemistry,” 6th Ed., Smith, M. B. and March, J., eds. John Wiley & Sons, New York: 2007, the entire contents of which are hereby incorporated by reference.

The term “hydrocarbon”, as used herein by itself or as part of a group, refers to a straight- or branched-chain aliphatic series of one to two hundred carbon atoms, i.e., a C1-C200 hydrocarbon, or the number of carbon atoms designated, e.g., a C1 hydrocarbon such as a methyl, a C2 hydrocarbon such as ethyl, etc. In one embodiment, the hydrocarbon is a C2-C200 hydrocarbon group. In an embodiment, the hydrocarbon is a C6-C60 hydrocarbon group. In an embodiment, the hydrocarbon is a C6-C60 hydrocarbon group. In an embodiment, the hydrocarbon is a C2-C60 hydrocarbon group. In an embodiment, the hydrocarbon is a C5-C22 hydrocarbon group. Examples of hydrocarbon groups include butyl, octyl, decyl, lauryl, cetyl (palmityl), and stearyl.

The term “alkyl”, as used herein by itself or as part of a group, refers to a straight or branched-chain aliphatic hydrocarbon containing one to two hundred carbon atoms, i.e., a C2-C200 alkyl, or the number of carbon atoms designated, e.g., a C1 alkyl such as methyl, a C2 alkyl such as ethyl, etc. In one embodiment, the alkyl is a C2-C200 alkyl group. In another embodiment, the alkyl is a C6-C60 alkyl group. In another embodiment, the alkyl is a C2-C60 alkyl group. In another embodiment, the alkyl is a C5-C22 alkyl group. Examples of alkyl group include butyl, octyl, decyl, lauryl, cetyl (palmityl), and stearyl.

The term “alkene”, as used herein by itself or as part of a group, refers to an alkyl group containing one, two, three, or more carbon-to-carbon double bonds. In one embodiment, the alkene group is a C2-C200 alkylene group. In another embodiment, the alkene group is a C6-C60 alkene group. In another embodiment, the alkene group is a C6-C60 alkene group. In another embodiment, the alkene group is a C2-C60 alkene group. In another embodiment, the alkene group is a C5-C22 alkene group.

The term “alkyne”, as used herein by itself or as part of a group, refers to an alkyl group containing one, two, three, or more carbon-to-carbon triple bonds. In another embodiment, the alkyne is a C2-C200 alkyne group.

The term “cyclic”, as used herein by itself or as part of a group, refers to a stable cyclic compound containing three or more atoms. In an embodiment, the cyclic is a C3-C200 cyclic group. In an embodiment, the cyclic is a C6-C60 cyclic group. In an embodiment, the cyclic is a C5-C22 cyclic group. Examples of cyclic compound include benzene, cyclopentane, and cyclohexane.

The term “heteroalkyl”, as used herein by itself or as part of a group, refers to a stable straight or branched chain alkyl radical containing two to two hundred carbon atoms and at least one heteroatom, which can be the same or different, selected from O, N, or S, wherein the sulfur atom(s) can optionally be oxidized. The heteroatoms can be placed at any interior position of the heteroalkyl group or at a position at which the heteroalkyl group is attached to the remainder of the molecule. In an embodiment, the heteroalkyl is a C6-C60 heteroalkyl group. In an embodiment, the heteroalkyl is a C2-C60 heteroalkyl group. Examples of heteroalkyl compound include succinyl, adipoyl, and sebacoyl.

The term “heteroalkene”, as used herein by itself or as part of a group, refers to a stable straight or branched chain alkene radical containing two to two hundred carbon atoms and at least one heteroatom, which can be the same or different, selected from O, N, or S, wherein the sulfur atom(s) can optionally be oxidized. The heteroatoms can be placed at any interior position of the heteroalkyl group or at a position at which the heteroalkyl group is attached to the remainder of the molecule. In an embodiment, the heteroalkene is a C6-C60 heteroalkene group. In an embodiment, the heteroalkene is a C2-C60 heteroalkene group. Examples of heteroalkene compound include oleoyl, ricinolyl, and linoleoyl.

The term “heteroalkyne”, as used herein by itself or as part of a group, refers to a stable straight or branched chain alkyne radical containing two to two hundred carbon atoms and at least one heteroatom, which can be the same or different, selected from O, N, or S, wherein the sulfur atom(s) can optionally be oxidized. The heteroatoms can be placed at any interior position of the heteroalkyl group or at a position at which the heteroalkyl group is attached to the remainder of the molecule. The term “heterocyclic”, as used herein by itself or as part of a group, refers to a stable cyclic compound containing three or more atoms and at least one heteroatom, which can be the same or different, selected from O, N, or S. In an embodiment, the heterocyclic is a C2-C200 heterocyclic group. In an embodiment, the heterocyclic is C6-C60 heterocyclic group.

The term “heterocyclic”, as used herein by itself or as part of a group, refers to a stable cyclic compound containing two or more carbons atoms and at least one heteroatom, which can be the same or different, selected from O, N, or S, wherein the sulfur atom(s) can optionally be oxidized. In an embodiment, the heterocyclic is a C2-C200 heterocyclic group. In an embodiment, the heterocyclic is a C6-C60 heterocyclic group. In an embodiment, the heterocyclic is a C5-C22 heterocyclic group. Examples of heterocyclic compounds include furan, oxolane, and thiophene.

As used herein, the term “olefin” refers to any species having at least one ethylenic double bond such as normal and branched chain aliphatic olefins, cycloaliphatic olefins, aryl substituted olefins, and the like. An olefin can comprise terminal double bond(s) (“terminal olefin”) and/or internal double bond(s) (“internal olefin”) and can be cyclic or acyclic, linear or branched, optionally substituted. The total number of carbon atoms can be from 1 to 100, or from 1 to 40; the double bonds can be unsubstituted or mono-, bi-, tri-or tetrasubstituted.

As used herein, the term “polyolefin” refers to a homopolymer or copolymer of ethylene, propylene, butenes and other unsaturated aliphatic hydrocarbons, vinyl esters (e.g. vinyl acetate), or (meth)acrylics (e.g. butyl acrylate, acrylic acid). Generally, the polyolefin will be a polymer of ethylene, propylene or copolymer thereof, or a copolymer of ethylene or propylene with one or more C4-C12 α-olefin aliphatic comonomers.

A gel is a disperse system comprising at least two components: a solid component and a liquid component. The solid component forms a sponge-like, three-dimensional network whose pores are filled by a liquid. The liquid component is thus immobilized in the solid. In the gel of the invention the solid component is a three-dimensional network formed of the cross-linked polyester elastomer, and the liquid component is formed of one or more solvents or emollients as defined herein. A gel is a semi-solid that can have properties ranging from soft and weak to hard and tough. Gels are also defined as a substantially dilute cross-linked system.

An elastomer gel is thus made from the elastomer powders or particles swelled or dispersed in a liquid, such as a solvent or an emollient, to form a gel. The ability to swell is commonly expressed by the swelling ratio as explained herein.

The term “skin care active agents (A)” used herein generally refers to specific ingredients or substances in skin care products that actively affect the skin's structure and function, typically by providing therapeutic or cosmetic benefits. These compounds are formulated to address various skin concerns such as aging, acne, pigmentation, dryness, sensitivity, sun damage, or the like. Active compounds can include e.g. vitamins, peptides, acids, antioxidants, and botanical extracts, among others. The “skin care active agents (A)” can be used alone or in combination of two or more in the compositions of the proposed formulations. The “skin care active agents (A)” can be any and include for example anti-blotching compounds, anti-aging compounds, eye contour compounds, slimming compounds, soothing/sunburn compounds, anti-irritating compounds, skin firming and lifting compounds, free radical scavenger compounds, hydrative compounds, vitamins, anti-oxidants, anti-perspirant and deodorant actives and minerals.

Common active compounds in skin care (A), can be categorized e.g. by their primary functions, such as

### 1. Hydrators and Humectants, e.g.

Hyaluronic Acid: Draws moisture into the skin and helps maintain hydration.
Glycerin: A humectant that attracts water to the skin, promoting moisture retention.
Propylene Glycol: A humectant that hydrates the skin by drawing water from the air.
Sodium PCA: A natural moisturizing factor (NMF) that binds water to the skin.
Urea: Moisturizes and softens the skin by enhancing water retention.

### 2. Exfoliants (Chemical Exfoliants) e.g.

Alpha Hydroxy Acids (AHAs), such as
   Glycolic Acid: Exfoliates and improves skin texture by removing dead skin cells.
   Lactic Acid: Gently exfoliates and promotes skin renewal while offering mild hydration.
   Citric Acid: Exfoliates and brightens the skin by sloughing off dead skin cells.
Beta Hydroxy Acid (BHAs), such as
   Salicylic Acid: A fat-soluble acid that penetrates pores to help treat acne and oily skin.
Polyhydroxy Acids (PHAs): Gentler than AHAs, PHAs exfoliate and hydrate the skin.
   Gluconolactone: An exfoliating agent with additional antioxidant properties.

### 3. Brightening Agents

Vitamin C (Ascorbic Acid): An antioxidant that helps brighten skin tone and reduce dark spots.
Niacinamide (Vitamin B3): Reduces hyperpigmentation and evens out skin tone.
Alpha Arbutin: A natural skin brightener that helps lighten dark spots and hyperpigmentation.
Licorice Extract: A natural extract that inhibits melanin production and brightens the skin.
Kojic Acid: A by-product of fungi used to treat hyperpigmentation and dark spots.

### 4. Anti-Aging Compounds

Retinoids (Vitamin A derivatives):
   Retinol: Reduces wrinkles and fine lines, promotes collagen production, and improves skin texture.
   Tretinoin (Retinoic Acid): A prescription-strength retinoid for treating acne, aging signs, and hyperpigmentation.
   Retinaldehyde: A gentler alternative to retinol, converting to retinoic acid in the skin.
Peptides:
   Palmitoyl Pentapeptide-4: Stimulates collagen production and reduces the appearance of wrinkles.
   Copper Peptides: Promote collagen synthesis and improve skin elasticity.

### 5. Anti-Inflammatory and Calming Ingredients

Centella Asiatica (Cica): Known for its healing and calming properties, it aids in repairing damaged skin and soothing irritation.
Aloe Vera: Hydrates and calms the skin while reducing inflammation and redness.
Green Tea Extract: Contains antioxidants and anti-inflammatory compounds that protect the skin and reduce redness.
Chamomile Extract: Soothes irritation and reduces inflammation.
Azelaic Acid: Reduces redness, acne, and rosacea while promoting skin renewal.

### 6. Acne Treatment Compounds

Benzoyl Peroxide: Targets acne-causing bacteria and helps prevent future breakouts.
Sulfur: Works as an antibacterial and helps reduce acne by drying out excess oils.
Tea Tree Oil: A natural antibacterial agent that helps reduce acne-causing bacteria.
Zinc: Reduces inflammation and regulates sebum production, making it beneficial for acne-prone skin.

### 7. Skin Barrier Support.

Ceramides: Lipid molecules that help restore and maintain the skin barrier.
Fatty Acids (e.g., Linoleic Acid): Help restore the skin's natural moisture barrier and reduce trans-epidermal water loss (TEWL).
Cholesterol: Works alongside ceramides to improve the skin barrier and enhance hydration.
Squalane: A natural emollient that moisturizes the skin and restores the barrier.

### 8. Antioxidants

Coenzyme Q10 (Ubiquinone): Protects the skin from oxidative stress and prevents premature aging.
Green Tea Polyphenols: Protect the skin from environmental damage and offer anti-inflammatory effects.
Resveratrol: A powerful antioxidant that combats signs of aging and protects the skin from free radicals.
Ferulic Acid: Enhances the stability and efficacy of Vitamin C and E in skincare.

### 9. Anti-Perspirant and Deodorant Actives (APDO).

At least one fragrance compound obtained from a source selected from the group consisting of essential oils, flower oils, natural extracts from resins, gums, balsams, beans, mosses, plants, ambergris, musk, synthetic aromatic materials and combinations thereof.
Antiperspirant actives such as Aluminum Zirconium Tetrachlorohydrex Gly, Aluminum Chlorohydrate, Aluminum Sesquichlorohydrate, Aluminum Zirconium Trichlorohydrex Gly.
Antiperspirant natural alternatives like Arrowroot Powder, Baking Soda (Sodium Bicarbonate), Kaolin Clay, Magnesium Hydroxide.

### 10. Probiotic, Postbiotic and Prebiotic Ingredients

One or more probiotics as a selective antibacterial ingredient obtained from a source such as those selected from Bifidobacteria longum 51A; Bifidobacteria bifidum 1622A; Bifidobacteria breve 1101A; Bifidobacteria pseudolongum 1191A, or a mixture thereof.
Lysates of probiotic microorganisms such as those selected from genus Bacillus, genus Bifidobacterium, genus Lactobacillus, genus Saccharomyces, genus Streptococcus, or a mixture thereof.
Prebiotic ingredients such as those selected from oligosaccharides like fructooligosaccharides (FOS) and galactooligosaccharides (GOS), inulin, glucose, fructose, xylitol, glucomannan, and rhamnose, and other prebiotics that can be found in plant extracts from sources such as chicory root, barley, oats, and flaxseed.

### 11. Other Active Ingredients

Alpha-Lipoic Acid: A powerful antioxidant that helps reduce wrinkles, improve skin texture, and fade hyperpigmentation.
Mandelic Acid: An AHA derived from almonds, it is gentler on the skin while offering exfoliation and brightening benefits.
Hesperidin: A flavonoid that improves blood circulation and can reduce under-eye puffiness.

These compounds can be applied various forms of the cosmetic compositions, such as serums, creams, masks, and toners, and are typically used depending on the specific skin concern being treated (e.g., acne, aging, hydration, pigmentation).

Suitable examples of skin care active compounds (A) include for example: whitening ingredients such as ascorbyl palmitate, kojic acid, lucinol, and oil-soluble licorice extract, wrinkle preventers such as retinol, retinoic acid, retinol acetate and retinol palmitate, circulation improving ingredients such as capsaicin and vanillylamide nonylate, antimicrobial ingredients such as isopropylmethylphenol, light-sensitive elements and zinc oxide, and vitamins such as vitamin D, vitamin D and vitamin K.

In an embodiment the compositions contain one or more humectants such as polyols such as glycerin and propylene glycol.

Cosmetically acceptable carriers (C) can be suitably selected from the group consisting of water, solvents, emollients, fatty acids, fatty alcohols, thickeners and combinations thereof. Preferably they are selected from the solvents and emollients as described herein below.

### DESCRIPTION OF THE FIGURES

FIG. 1 is a line graph showing the particle size distribution (determined as described herein) of the polyester elastomer gel prepared by processing the polyester elastomer of Example 1 with coco-caprylate/caprate solvent or emollient.

FIG. 2 shows the controlled release profile of retinol from a composition containing polyester elastomer (B) comprising retinol (A) compared to a control 1 (replacing polyester gel (B) by isododecane) and a control 2 (replacing polyester elastomer (B) by a silicon elastomer), as measured with a membrane placed in a Franz diffusion cell as described in example 6.

FIGS. 3 and 4 show the controlled release profiles of niacinamide from a W/O and an O/W composition containing polyester elastomer (B) comprising Niacinamide (A) each compared to a control 1 (replacing polyester gel (B) by isododecane) and a control 2 (replacing polyester elastomer (B) by a silicon elastomer), as measured with a membrane placed in a Franz diffusion cell as described in example 7.

FIG. 5 shows the result of stability tests of a composition containing polyester elastomer (B) comprising Vitamin C (component (A)) and a composition comprising Vitamin C (component (A)) which does not contain the polyester elastomer (B).

FIG. 6 shows the cumulative release profile of Retinol from water-in-oil formulation containing polyester elastomer (B) comprising Retinol (A) of Example 6.

FIG. 7 shows the cumulative release profile of Niacinamide from a water-in-oil formulation containing polyester elastomer (B) comprising Niacinamide (A) of Example 7.

FIG. 8 shows the cumulative release profile of Niacinamide from an oil-in-water formulation containing polyester elastomer (B) comprising Niacinamide (A) of Example 8

FIG. 9 shows the cumulative release profile of Niacinamide from the formulation, containing different concentrations of the polyester elastomer (B), comprising Niacinamide (A) of Example 9.

FIG. 10 shows the simultaneous release of Retinol and Niacinamide from the formulation containing polyester elastomer (B) comprising Niacinamide (A) and Retinol (A) of Example 9.

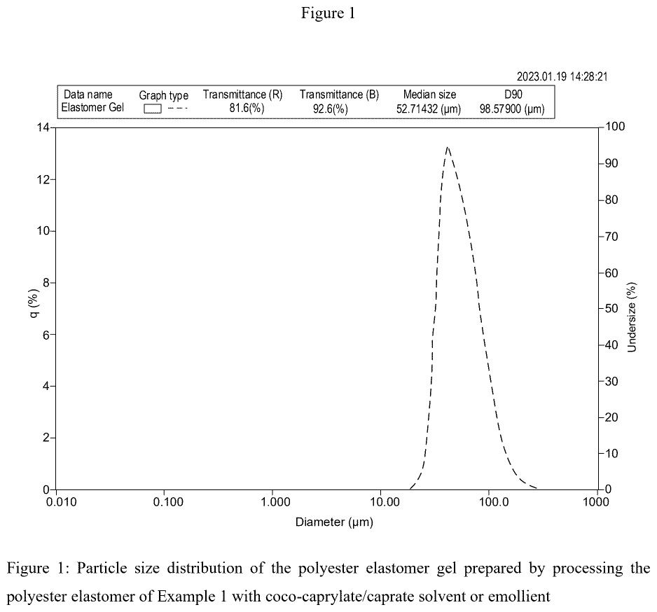

### A. Components of the Crosslinked Polyester Component (B)

### 1. Poly-Carboxylic Acids (i)

In some aspects, the at least one poly-carboxylic acid may be a compound of formula (VI)

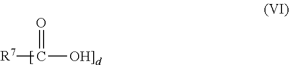

wherein
   R⁷ is C2-C200 alkyl group, C2-C200 heteroalkyl group, C2-C200 alkene group, C2-C200 heteroalkene group, C2-C200 alkyne group, C2-C200 heteroalkyne group, C3-C200 cyclic group, or C2-C200 heterocyclic group; and
   d is an integer from 2 to 10.

In a further aspect, the compound is formula (I), wherein R¹ is C6-C60 alkyl group, C6-C60 heteroalkyl group, C6-C60 alkene group, C6-C60 heteroalkene group, C6-C60 cyclic group, or C6-C60 heterocyclic group; and a is an integer from 2 to 10. In an aspect, the compound is formula (I), wherein a is 2, 3, 4, 5, 6, 7, 8, 9, or 10. In an aspect, the compound is formula (I), wherein a is an integer from 2 to 6. In another aspect, the compound is formula (I), wherein a is 2, 3, 4, 5, or 6.

In an aspect, the polycarboxylic acid is a product of a compound of formula (I), and/or a compound of formula (III) with a compound of formula (V).

In an aspect, the at least one polycarboxylic acid may be selected from the group consisting of citric acid, isocitric acid, aconitic acid, propane-1,2,3-tricarboxylic acid, trimesic acid, carballylic acid, C54 trimer acid, mellitic acid, and combinations thereof. In a further aspect, the at least one polycarboxylic acid may be selected from the group consisting of citric acid, C54 trimer acid, and combinations thereof.

In an aspect, C36 dimer acid is a dicarboxylic acid prepared by dimerizing unsaturated linoleic fatty acid from plant oil.

In an aspect, C54 trimer acid is a polycarboxylic acid prepared by trimerizing unsaturated fatty acids from plant oil.

In some aspects, the unsaturated fatty acids are palmitoleic acid, oleic acid, elaidic acid, vaccenic acid, linoleic acid, linoelaidic acid, or linolenic acid.

In some aspects, the plant oils are soybean oil, safflower oil, linseed oil, corn oil, sunflower oil, olive oil, canola oil, sesame oil, cottonseed oil, palm oil, rapeseed oil, tung oil, peanut oil, or milkweed oil.

In an aspect, the at least one carboxylic acid may be a dicarboxylic acid. In an aspect, the dicarboxylic acid is a compound of formula (VII)

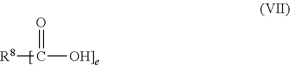

wherein
   R⁸ is C2-C200 alkyl group, C2-C200 heteroalkyl group, C2-C200 alkylene group, C2-C200 heteroalkylene group, C2-C200 alkyne group, C2-C200 heteroalkyne group, C3-C200 cyclic group, or C2-C200 heterocyclic group; and
   e is 2.

In another aspect, the compound is formula (VII), wherein R⁸ is C2-C200 alkyl group, C2-C200 heteroalkyl group, C2-C200 alkene group, or C2-C200 heteroalkene group and e is 2. In a further aspect, the compound is of formula (VII), wherein R⁸ is C2-C60 alkyl group, C2-C60 heteroalkyl group, C2-C60 alkene group, or C2-C60 heteroalkene group and e is 2. In an aspect, the compound is of formula (VII), wherein R⁸ may be succinyl, adipoyl, sebacoyl, dilinoleyl, or trilinoleyl.

In an aspect, the compound is formula (VII), wherein e may be 1 or 2.

In some aspects, the dicarboxylic acid may be selected from the group consisting of malonic acid, succinic acid, fumaric acid, glutaric acid, adipic acid, pimelic acid, suberic acid, azelaic acid, sebacic acid, undecanedioic acid, dodecanedioic acid, tridecanedioic acid, hexadecanedioic acid, C21 dimer acid, C36 dimer acid, hydrogenated C36 dimer acid, aspartic acid, glutamic acid, tartaric acid, malic acid, and combinations thereof. In a further aspect, the dicarboxylic acid may be selected from the group consisting of malonic acid, succinic acid, adipic acid, azelaic acid, sebacic acid, undecanedioic acid, dodecanedioic acid, C21 dimer acid, C36 dimer acid, hydrogenated C36 dimer acid, and combinations thereof. In one aspect, the dicarboxylic acid is selected from the group consisting of succinic acid, adipic acid, azelaic acid, sebacic acid, undecanedioic acid, dodecanedioic acid, C21 dimer acid, C36 dimer acid, hydrogenated C36 dimer acid, and combinations thereof.

In an aspect, the poly-carboxylic acid is biobased or naturally derived.

### 2. Polycarboxylic Acid Ester (i)

In an aspect, the at least one polycarboxylic acid ester may be a compound of formula (VIII)

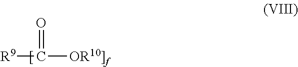

wherein
   R⁹ is C1-C22 alkyl group, C2-C22 alkylene group, or C3-C22 cyclic group;
   R¹⁰ is C2-C200 alkyl group, C2-C200 heteroalkyl group, C2-C200 alkene group, C2-C200 heteroalkene group, C2-C200 alkyne group, C2-C200 heteroalkyne group, C2-C200 cyclic group, or C2-C200 heterocyclic group; and
   f is an integer from 2 to 10.

In another aspect, the compound is formula (VIII), wherein R⁹ is C1-C22 alkyl group or C2-C22 alkene group; R¹⁰ is C2-C200 alkyl group, C2-C200 heteroalkyl group, C2-C200 alkene group, or C2-C200 heteroalkene group; and f is an integer from 3 to 10.

In a further aspect, the compound is formula (VIII), wherein R⁹ is C1-C10 alkyl group; R¹⁰ is C2-C60 alkyl group, C2-C60 heteroalkyl group, C2-C60 alkene group, or C2-C60 heteroalkene group; and f is an integer from 3 to 10.

In an aspect, the compound is formula (VIII), wherein f is an integer from 3 to 6. In an aspect, the compound is formula (VIII), wherein f is 3, 4, 5, or 6.

In an aspect, the polycarboxylic acid ester is a product of a compound of formula (I), and/or a compound of formula (III) with a compound of formula (V).

In some aspects, the polycarboxylic acid ester may be selected from the group consisting of triethyl citrate, triethyl isocitrate, aconitic acid triethyl ester, propane-1,2,3-tricarboxylic acid triethyl ester, trimesic acid triethyl ester, carballylic acid triethyl ester, C54 trimer acid triethyl ester, mellitic acid hexaethyl ester, and combinations thereof. In a further aspect, the polycarboxylic acid ester may be selected from the group consisting of triethyl citrate, C54 trimer acid triethyl ester, and combinations thereof.

In an aspect, the at least one polycarboxylic acid ester may be a dicarboxylic acid ester. In some aspects, the dicarboxylic acid ester may be a compound of formula (IX)

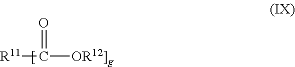

wherein
   R¹¹ is C1-C22 alkyl group, C2-C22 alkene group, or C3-C22 cyclic group;
   R¹² is C2-C200 alkyl group, C2-C200 heteroalkyl group, C2-C200 alkylene group, C2-C200 heteroalkene group, C2-C200 alkyne group, C2-C200 heteroalkyne group, C3-C200 cyclic group, or C2-C200 heterocyclic group; and
   g is 2.

In an aspect, the compound is formula (IX), wherein R¹¹ is C1-C22 alkyl group or C2-C22 alkylene group; R¹² is C2-C200 alkyl group, C2-C200 heteroalkyl group, C2-C200 alkene group, or C2-C200 heteroalkene group; and g is 2. In a further aspect, the compound is formula (IX), wherein R¹¹ is C1-C10 alkyl group; R¹² is C2-C60 alkyl group, C2-C60 heteroalkyl group, C2-C60 alkene group, or C2-C60 heteroalkene group; and g is 2.

In some aspects, the dicarboxylic acid may be selected from the group consisting of diethyl malonate, diethyl succinate, diethyl fumarate, diethyl adipate, diethyl pimelate, diethyl suberate, diethyl azelate, diethyl sebacate, diethyl undecanedioate, diethyl dodecanedioate, diethyl tridecanedioate, diethyl hexadecanedioiate, C21 dimer acid diethyl ester, C36 dimer acid diethyl ester, hydrogenated C36 dimer acid diethyl ester, diethyl aspartate, diethyl glutamate, diethyl tartrate, diethyl malate, and combinations thereof. In a further aspect, the dicarboxylic acid ester may be selected from the group consisting of diethyl malonate, diethyl succinate, diethyl adipate, diethyl pimelate, diethyl azelate, diethyl sebacate, diethyl undecanedioate, C21 dimer acid diethyl ester, C36 dimer acid diethyl ester, hydrogenated C36 dimer acid diethyl ester, and combinations thereof.

In an aspect, the poly-carboxylic acid ester is biobased or naturally derived.

### 3. Polyol (ii)

In an aspect, the at least one polyol is a compound of formula (X)

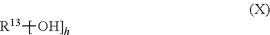

wherein
   R¹³ is C2-C200 alkyl group, C2-C200 heteroalkyl group, C2-C200 alkene group, C2-C200 heteroalkene group, C2-C200 alkyne group, C2-C200 heteroalkyne group, C3-C200 cyclic group, or C2-C200 heterocyclic group; and
   h is an integer from 2 to 10.

In an aspect, the compound is formula (X), wherein R¹³ is C2-C200 alkyl group, C2-C200 heteroalkyl group, C2-C200 alkene group, or C2-C200 heteroalkene group; and h is an integer from 2 to 10. In a further aspect, the compound is formula (X), wherein R¹³ is C2-C60 alkyl group, C2-C60 heteroalkyl group, C2-C60 alkene group, or C2-C60 heteroalkene group; and h is an integer from 2 to 10.

In an aspect, the compound is formula (X), wherein h is an integer from 2 to 6. In an aspect, the compound is formula (X), wherein h is 2, 3, 4, 5, or 6.

In an aspect, the polyol is a product of a compound of formula (I), and/or a compound of formula (III) with a compound of formula (V).

In an aspect, the C36 dimer diol is the diol produced from a C36 dimer acid.

In some aspects, the polyol may be selected from the group consisting of glycerol, diglycerol, polyglycerol, sorbitan, castor oil, hydrogenated castor oil, sugar alcohol, monosaccharide, disaccharides, oligosaccharide, polysaccharides, tannin, gallic acid, gluconic acid, lactobionic acid, gluconolactone, and combinations thereof. In a further aspect, the polyol may be selected from the group consisting of glycerol, diglycerol, polyglycerol, castor oil, hydrogenated castor oil, sorbitol, gallic acid, and combinations thereof. In another aspect, the alcohol may be selected from the group consisting of glycerol, diglycerol, polyglycerol, castor oil, hydrogenated castor oil, sorbitol, and combinations thereof.

In some aspects, the at least one polyol is a diol. In some aspects, the diol is a compound of formula (XI)

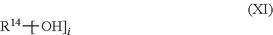

wherein
   R¹⁴ is C2-C200 alkyl group, C2-C200 heteroalkyl group, C2-C200 alkene group, C2-C200 heteroalkene group, C2-C200 alkyne group, C2-C200 heteroalkyne group, C3-C200 cyclic group, or C2-C200 heterocyclic group; and
   i is 2.

In an aspect, the compound is formula (XI), wherein R¹⁴ is C2-C200 alkyl group, C2-C200 heteroalkyl group, C2-C200 alkene group, or C2-C200 heteroalkene group; and i is 2. In a further aspect, the compound is formula (XI), wherein R¹⁴ is C2-C60 alkyl group, C2-C60 heteroalkyl group, C2-C60 alkene group, or C2-C60 heteroalkene group; and i is 2.

In some aspects, the diol may be selected from the group consisting of ethyleneglycol, 1,2-propanediol, 1,3-propanediol, 1,3-butanediol, 1,4-butanediol, 1,2-pentanediol, 1,3-pentanediol, 1,4-pentanediol, 1,5-pentanediol, 1,2-hexanediol, 1,5-hexanediol, 1,6-hexanediol, C36 dimer diol, and combinations thereof. In a further aspect, the diol may be selected from the group consisting of 1,3-propanediol, 1,3-butanediol, 1,4-butanediol, 1,5-pentanediol, 1,6-hexanediol, C36 dimer diol, hydrogenated C36 dimer diol, and combinations thereof. In one aspect, the diol may be selected from the group consisting of 1,3-propanediol, 1,3-butanediol, 1,4-butanediol, 1,5-pentanediol, 1,6-hexanediol, C36 dimer diol, hydrogenated C36 dimer diol, and combinations thereof.

In an aspect, the polyol is biobased or naturally derived.

4. Mono-carboxylic Acids (iii) (a)

In an aspect, the at least one mono-carboxylic acid is a compound of formula (XII)

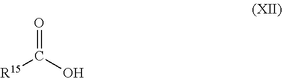

wherein
   R¹⁵ is C2-C52 alkyl group, C2-C52 heteroalkyl group, C2-C52 alkene group, C2-C52 heteroalkene group, C3-C52 cyclic group, or C2-C52 heterocyclic group.

In an aspect, the mono-carboxylic acid is a compound of formula (XII), wherein R¹⁵ is C5-C21 alkyl group, C5-C21 heteroalkyl group, C5-C21 alkene group, C5-C21 heteroalkene group, C5-C21 cyclic group, or C5-C21 heterocyclic group.

In an aspect, the mono-carboxylic acid is selected from the group consisting of caprylic acid, capric acid, lauric acid, myristic acid, palmitic acid, stearic acid, isostearic acid, behenic acid, oleic acid, linoleic acid, linolenic acid, and combinations thereof. In an aspect, the mono-carboxylic acid is oleic acid. In an aspect, the mono-carboxylic acid is isostearic acid.

In an aspect, R¹⁵ is C2-C52 alkyl group with at least one hydroxyl (—OH) group.

In an aspect, the mono-carboxylic acid is selected from the group consisting of hydroxylstearic acid, ricinoleic acid, isoricinoleic acid, lesquerolic acid, densipolic acid, auricolic acid, dimorphecolic acid, and combinations thereof. In an aspect, the mono-carboxylic acid is hydroxylstearic acid, ricinoleic acid, and combinations thereof.

In an aspect, the mono-carboxylic acid is biobased or naturally derived.

5. Mono-alcohols (iii) (b)

In an aspect, the at least one mono-alcohol is a compound of formula (XIII)

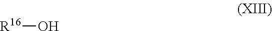

wherein
   R¹⁶ is C2-C52 alkyl group, C2-C52 heteroalkyl group, C2-C52 alkene group, C2-C52 heteroalkene group, C3-C52 cyclic group, or C2-C52 heterocyclic group.

In an aspect, the mono-alcohol is a compound of formula (XIII), wherein R¹⁶ is C5-C21 alkyl group, C5-C21 heteroalkyl group, C5-C21 alkene group, C5-C21 heteroalkene group, C5-C21 cyclic group, or C5-C21 heterocyclic group.

In an aspect, the mono-alcohol is selected from the group consisting of octanol, decanol, lauryl alcohol, myristyl alcohol, cetyl alcohol, stearyl alcohol, isostearyl alcohol, behenyl alcohol, oleyl alcohol, linoleyl alcohol, linolenyl alcohol, and combinations thereof. In an aspect, the mono-alcohol is oleyl alcohol. In an aspect, the mono-alcohol is isostearyl alcohol.

In an aspect, the mono-alcohol is biobased or naturally derived.

### B. Ratio of Components for the Preparation of Crosslinked Polyester Component (B)

In an aspect, the elastomer comprises a defined molar ratio of carboxyl functional groups (—COOH) from mono-carboxylic acid to hydroxyl functional groups (—OH) from polyol, and from hydroxyl functional groups (—OH) from mono-alcohol to carboxyl functional groups (—COOH) from poly-carboxylic acid. It has been found that the molar ratio of carboxyl functional groups (—COOH) from mono-carboxylic acid to hydroxyl functional groups (—OH) from the polyol and the molar ratio of hydroxyl functional groups (—OH) from mono-alcohol to carboxyl functional groups (—COOH) from poly-carboxylic acid influences the performance of the polyester elastomer and the performance of the gel made from the polyester elastomer.

In an aspect, the molar ratio of carboxyl functional groups (—COOH) from monocarboxylic acid to hydroxyl functional groups (—OH) from polyol is from about 1:2 to about 1:16. In an aspect, the molar ratio of carboxyl functional groups (—COOH) from mono-carboxylic acid to hydroxyl functional groups (—OH) from polyol is from about 1:2 to about 1:14. In an aspect, the molar ratio of carboxyl functional groups (—COOH) from mono-carboxylic acid to hydroxyl functional groups (—OH) from polyol is from about 1:2 to about 1:10. In an aspect, the molar ratio of carboxyl functional groups (—COOH) from mono-carboxylic acid to hydroxyl functional groups (—OH) from polyol is from about 1:2 to about 1:8. In an aspect, the molar ratio of carboxyl functional groups (—COOH) from mono-carboxylic acid to hydroxyl functional groups (—OH) from polyol is from about 1:2 to about 1:5. In an aspect, the molar ratio of carboxyl functional groups (—COOH) from mono-carboxylic acid to hydroxyl functional groups (—OH) from polyol is about 1:16, about 1:15, about 1:14, about 1:13, about 1:12, about 1:11, about 1:10, about 1:9, about 1:8, about 1:7, about 1:6, about 1:5, about 1:4, about 1:3, or about 1:2.

In an aspect, the molar ratio of hydroxyl functional groups (—OH) from mono-alcohol to carboxyl functional groups (—COOH) from poly-carboxylic acid is from about 1:2 to about 1:16. In an aspect, the molar ratio of hydroxyl functional groups (—OH) from mono-alcohol to carboxyl functional groups (—COOH) from poly-carboxylic acid is from about 1:2 to about 1:14. In an aspect, the molar ratio of hydroxyl functional groups (—OH) from mono-alcohol to carboxyl functional groups (—COOH) from poly-carboxylic acid is from about 1:2 to about 1:10. In an aspect, the molar ratio of hydroxyl functional groups (—OH) from mono-alcohol to carboxyl functional groups (—COOH) from poly-carboxylic acid is from about 1:2 to about 1:8. In an aspect, the molar ratio of hydroxyl functional groups (—OH) from mono-alcohol to carboxyl functional groups (—COOH) from poly-carboxylic acid is from about 1:2 to about 1:5. In an aspect, the molar ratio of hydroxyl functional groups (—OH) from mono-alcohol to carboxyl functional groups (—COOH) from poly-carboxylic acid is about 1:16, about 1:15, about 1:14, about 1:13, about 1:12, about 1:11, about 1:10, about 1:9, about 1:8, about 1:7, about 1:6, about 1:5, about 1:4, about 1:3, or about 1:2.

In an aspect, the molar ratio of carboxyl functional groups (—COOH) from poly-carboxylic acid to hydroxyl functional groups (—OH) from polyol is from about 1.5:1 to about 1:4. In an aspect, the ratio of carboxyl functional groups (—COOH) from poly-carboxylic acid to hydroxyl functional groups (—OH) from polyol is from about 1.5:1 to about 1:2. In an aspect, the ratio of carboxyl functional groups (—COOH) from poly-carboxylic acid to hydroxyl functional groups (—OH) from polyol is from about 1.5:1 to about 1:1.5. In an aspect, the ratio of carboxyl functional groups (—COOH) from poly-carboxylic acid to hydroxyl functional groups (—OH) from polyol is from about 1.5:1 to about 1:1.25. In an aspect, the ratio of carboxyl functional groups (—COOH) from poly-carboxylic acid to hydroxyl functional groups (—OH) from polyol is about 1.5:4, about 1.5:3, about 1.5:2, about 1:1, about 1:2, about 1:3, or about 1:4.

In an aspect, the conversion of carboxylic acid functional groups (—COOH) to ester functional groups (—CO(O)—) is no less than 80% by mole. The percent conversion is calculated by titration of carboxylic acid functional groups (—COOH) with 0.1 N KOH in isopropanol.

### C. Methods of Preparing the Polyester Elastomer (B)

### 1. Esterification Reaction

In one aspect, the present disclosure is directed to a method of preparing an elastomer (A) comprising reacting:
(i) at least one poly-carboxylic acid, at least one poly-carboxylic acid ester, or a combination thereof; and
(ii) at least one polyol; and
(iii) optionally one or more mono-carboxylic acids; and/or
(iv) optionally one or more mono-alcohols.

In an aspect, the elastomer prepared is a polyester elastomer (A). In an aspect, the elastomer prepared is a crosslinked polyester elastomer.

In an aspect, the preparation of the elastomer (A) is under nitrogen protection, is under vacuum, or is a combination thereof.

In an aspect, the elastomer is prepared by reacting:
(i) at least one poly-carboxylic acid, at least one poly-carboxylic acid ester, or a combination thereof; and
(ii) at least one polyol; and
(iii) at least one mono-carboxylic acid; and/or
(iv) at least one mono-alcohol.

In an aspect, the crosslinked polyester component (A) is reaction product of:
(i) one or more poly-carboxylic acids selected from di-carboxylic acids, tri-carboxylic acids and combinations thereof;
(ii) one or more polyols selected from diols, triols and combinations thereof, and
(iii) (a) optionally one or more monofunctional carboxylic acids.

In an aspect, the crosslinked polyester (A) is reaction product of:
(i) one or more dicarboxylic acids
(ii) one or more triols, and
(iii) (a) optionally one or more monofunctional carboxylic acids.

In an aspect, wherein the reaction comprised an activated di-carboxylic acid or tricarboxylic acid, the preparation of the elastomer further comprises addition of water to quench the activating agent from the reaction.

In an aspect, esterification is conducted in solvent or emollient. In another aspect, esterification is conducted in more than one solvent or emollient.

In an aspect, esterification is carried out in the absence of a solvent or emollient.

In an aspect the crosslinked polyester component (B) is obtained by a process comprising the steps of:
(a) reacting compounds (i), (ii) and optionally (iii) as defined in the claims,
   optionally in the presence of one or more solvents
   optionally in the presence of one or more skin care active compound (A),
to obtain a cross-linked polyester or a mixture thereof with the optional components,
(b) optionally subjecting the crosslinked polyester or a mixture thereof with the optional components obtained in step (a) to a shearing process to obtain a cross-linked polyester component (B) or a mixture thereof with the optional components, or
(c) optionally adding one or more solvents to the crosslinked polyester or a mixture thereof with the optional components obtained in step (a), allowing the resulting mixture to swell in said solvent, subjecting said swollen mixture to a shearing process, a mechanical comminution process and/or a homogenization process in a reactor with planetary mixers to obtain a cross-linked polyester component (B) as a gel in said solvent or a mixture thereof with the optional components.

In an aspect a composition according to the previous paragraph, wherein the shearing process in steps (b) or (c) is carried out with a batch mixer, planetary mixer, single or multiple screw extruder, dynamic or static mixer, colloid mill, homogenizer, sonolator, three roll mill, or a combination thereof, preferably with a three-roll mill.

In an aspect step (b) is carried out by mechanical stirring. In an aspect the mechanical comminution process step (c) is carried out with a three-roll mill.

In an aspect the crosslinked polyester component (B) is obtained by the process, wherein step a) is carried out in the presence of one or more skin care active compounds (A).

In an aspect the amount of solvent added in step (c) above is such that the resulting composition used as crosslinked polyester component (B) comprises from about 10% to about 95%, preferably about 20% to about 70% or about 20% to about 50% weight by weight of the solvent, based on the total amount of the resulting composition. In a further aspect, the at least one di-carboxylic acid is a compound of formula (IA)

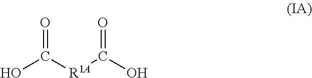

wherein
   R^{1A} is C2-C52 alkyl group, C2-C52 heteroalkyl group, C2-C52 alkene group, C2-C52 heteroalkene group, C3-C52 cyclic group, or C2-C52 heterocyclic group.

In an aspect, the di-carboxylic acid is a compound of formula (IA), wherein R^{1A} is C4-C34 alkyl group, C4-C34 heteroalkyl group, C4-C34 alkene group, C4-C34 heteroalkene group, C4-C34 cyclic group, or C4-C34 heterocyclic group.

In an aspect, the di-carboxylic acid is a compound of formula (IA), wherein RIA is C4-C34 alkyl group.

In an aspect, the di-carboxylic acid is selected from the group consisting of succinic acid, glutaric acid, adipic acid, pimelic acid, suberic acid, azelaic acid, sebacic acid, brassylic acid, dodecanedioic acid, C21 dimer acid, C36 dimer acid, maleic acid, fumaric acid, traumatic acid, and combinations thereof. In an aspect, the di-carboxylic acid is dilinoleic acid. In an aspect, the di-carboxylic acid is a C36 dimer acid. In an aspect, the di-carboxylic acid is a hydrogenated C36 dimer acid.

In an aspect, the di-carboxylic acid is biobased or naturally derived.

In an aspect, the at least one tri-carboxylic acid is a compound of formula (IB)

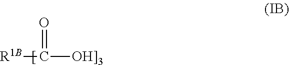

wherein
   R^{1B} is C2-C52 alkyl group, C2-C52 heteroalkyl group, C2-C52 alkene group, C2-C52 heteroalkene group, C3-C52 cyclic group, or C2-C52 heterocyclic group.

In an aspect, the tri-carboxylic acid is a compound of formula (IB), wherein R^{1B} is C4-C34 alkyl group, C4-C34 heteroalkyl group, C4-C34 alkene group, C4-C34 heteroalkene group, C4-C34 cyclic group, or C4-C34 heterocyclic group.

In an aspect, the tri-carboxylic acid is a compound of formula (IB), wherein R^{1B} is C4-C34 alkyl group.

In an aspect, the tri-carboxylic acid is selected from the group consisting of citric acid, C54 trimer acid, and hydrogenated C54 trimer acid. In an aspect, the tri-carboxylic acid is a C54 trimer acid.

In an aspect, the di-carboxylic acid is biobased or naturally derived.

In an aspect, the at least one mono-carboxylic acid is a compound of formula (IIB)

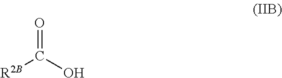

wherein
   R^{2B} is C2-C52 alkyl group, C2-C52 heteroalkyl group, C2-C52 alkene group, C2-C52 heteroalkene group, C3-C52 cyclic group, or C2-C52 heterocyclic group.

In an aspect, the mono-carboxylic acid is a compound is formula (II), wherein R^{2B} is C5-C21 alkyl group, C5-C21 heteroalkyl group, C5-C21 alkene group, C5-C21 heteroalkene group, C5-C21 cyclic group, or C5-C21 heterocyclic group.

In an aspect, the mono-carboxylic acid is selected from the group consisting of caprylic acid, capric acid, lauric acid, myristic acid, palmitic acid, stearic acid, isostearic acid, behenic acid, oleic acid, linoleic acid, linolenic acid, and combinations thereof. In an aspect, the mono-carboxylic acid is oleic acid. In an aspect, the mono-carboxylic acid is isostearic acid.

In an aspect, the mono-carboxylic acid is biobased or naturally derived.

In an aspect, the at least one polyol is a compound of formula (IIIB)

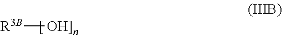

wherein
   R^{3B} is C3-C50 alkyl group, C3-C50 heteroalkyl group, C3-C50 alkene group, C3-C50 heteroalkene group, C3-C50 cyclic group, or C3-C50 heterocyclic group; and n is an integer from 2 to 10.

In an aspect, the polyol is a compound of formula (IIIB), wherein R^{3B} is C3-C50 alkyl group, C3-C50 heteroalkyl group, C3-C50 alkene group, or C3-C50 heteroalkene group; and n is an integer from 2 to 10.

In an aspect, the polyol is a compound of formula (IIIB), wherein R^{3B} is C3-C20 alkyl group, C3-C20 heteroalkyl group, C3-C20 alkene group, or C3-C20 heteroalkene group; and n is an integer from 2 to 10.

In an aspect, the polyol is a compound of formula (IIIB), wherein n is an integer from 2 to 6. In an aspect, the polyol is a compound of formula (IIIB), wherein n is 2, 3, 4, 5, or 6. In an aspect, the polyol is a compound of formula (IIIB), wherein n is an integer from 3 to 10. In an aspect, the polyol is a compound of formula (IIIB), wherein n is an integer from 3 to 6.

In an aspect, the polyol is selected from the group consisting of glycerol, diglycerol, polyglycerol, polyglycerol-3, sorbitol, castor oil, hydrogenated castor oil, sugar alcohol, monosaccharide, disaccharides, oligosaccharide, polysaccharides, tannin, gallic acid, 1,2-propanediol, 1,3-propanediol, 1,4-butanediol, 1,2-pentanediol, 1,3-pentanediol, 1,4-pentanediol, 1,5-pentanediol, 1,2-hexanediol, 1,5-hexanediol, 1,6-hexanediol, C36 dimer diol, hydrogenated C36 dimer diol, and combinations thereof. In an aspect, the polyol is hydrogenated castor oil. In an aspect, the polyol is diglycerol. In an aspect, the polyol is polyglycerol-3. In an aspect, the polyol is polyglycerol-4.

In an aspect, the polyol is biobased or naturally derived.

In principle, there are several options to making the compositions of the invention include for example:
1. Mixing (A)+ (B), where component (B) is made in the presence of a skin care active (A).
2. Mixing (A)+ (B), where component (B) is made in the absence of a solvent.
3. Mixing (A)+ (B), where component (B) is made in the presence of a solvent.
4. Preparing (B) in the presence of a skin care active (A) (no solvent).
5. Preparing (B) in the presence of a skin care active (A) and a solvent.
6. Preparing (B) in the presence of a skin care active (A) and another ingredient(s) commonly used in cosmetics (D) (no solvent).
7. Preparing (B) in the presence of a skin care active (A), a solvent and another ingredient(s) commonly used in cosmetics (D).

### 1. Ratio of Components

In an aspect, the preparation of the elastomer comprises a defined ratio of poly-carboxylic acid, poly-carboxylic acid ester or a combination thereof (i) to polyol (ii), a defined ratio of monocarboxylic acid (iii) (a) to polyol (ii), and a defined ratio of mono-alcohol (iii) (b) to poly-carboxylic acid, poly-carboxylic acid ester, or a combination thereof (i). It has been found that the ratio of (i)/(iii) (a), (ii)/(iii) (a), and (iii) (b)/(i) influences the performance of the polyester elastomer and the performance of the gel made from the polyester elastomer.

In an aspect, the molar ratio of carboxyl functional groups (—COOH) from monocarboxylic acid (iii) (a) to hydroxyl functional groups (—OH) from polyol (ii) is from about 1:2 to about 1:16. In an aspect, the molar ratio of carboxyl functional groups (—COOH) from mono-carboxylic acid (iii) (a) to hydroxyl functional groups (—OH) from polyol (ii) is from about 1:2 to about 1:14. In an aspect, the molar ratio of carboxyl functional groups (—COOH) from mono-carboxylic acid (iii) (a) to hydroxyl functional groups (—OH) from polyol (ii) is from about 1:2 to about 1:10. In an aspect, the molar ratio of carboxyl functional groups (—COOH) from mono-carboxylic acid (iii) (a) to hydroxyl functional groups (—OH) from polyol (ii) is from about 1:2 to about 1:8. In an aspect, the molar ratio of carboxyl functional groups (—COOH) from mono-carboxylic acid (iii) (a) to hydroxyl functional groups (—OH) from polyol (ii) is from about 1:2 to about 1:5. In an aspect, the molar ratio of carboxyl functional groups (—COOH) from mono-carboxylic acid (iii) (a) to hydroxyl functional groups (—OH) from polyol (ii) is about 1:16, about 1:15, about 1:14, about 1:13, about 1:12, about 1:11, about 1:10, about 1:9, about 1:8, about 1:7, about 1:6, about 1:5, about 1:4, about 1:3, or about 1:2.

In an aspect, the molar ratio of carboxyl functional groups (—COOH) from poly-carboxylic acid, poly-carboxylic acid ester or a combination thereof (i) to hydroxyl functional groups (—OH) from polyol (ii) is from about 1.5:1 to about 1:4. In an aspect, the ratio of carboxyl functional groups (—COOH) from poly-carboxylic acid, poly-carboxylic acid ester or a combination thereof (i) to hydroxyl functional groups (—OH) from polyol (ii) is from about 1.5:1 to about 1:2. In an aspect, the ratio of carboxyl functional groups (—COOH) poly-carboxylic acid, poly-carboxylic acid ester or a combination thereof (i) to hydroxyl functional groups (—OH) from polyol (ii) is from about 1.5:1 to about 1:1.5. In an aspect, the ratio of carboxyl functional groups (—COOH) poly-carboxylic acid, poly-carboxylic acid ester or a combination thereof (i) to hydroxyl functional groups (—OH) from polyol (ii) is from about 1.5:1 to about 1:1.25. In an aspect, the ratio of carboxyl functional groups (—COOH) poly-carboxylic acid, poly-carboxylic acid ester or a combination thereof (i) to hydroxyl functional groups (—OH) from polyol (ii) is about 1.5:4, about 1.5:3, about 1.5:2, about 1:1, about 1:2, about 1:3, or about 1:4.

In an aspect, the molar ratio of hydroxyl functional groups (—OH) from mono-alcohol (iii) (b) to carboxyl functional groups (—COOH) from poly-carboxylic acid (i) is from about 1:2 to about 1:16.

In an aspect, the molar ratio of hydroxyl functional groups (—OH) from mono-alcohol (iii) (b) to carboxyl functional groups (—COOH) from poly-carboxylic acid (i) is from about 1:2 to about 1:14. In an aspect, the molar ratio of hydroxyl functional groups (—OH) from mono-alcohol (iii) (b) to carboxyl functional groups (—COOH) from poly-carboxylic acid (i) is from about 1:2 to about 1:10. In an aspect, the molar ratio of hydroxyl functional groups (—OH) from mono-alcohol (iii) (b) to carboxyl functional groups (—COOH) from poly-carboxylic acid (i) is from about 1:2 to about 1:8. In an aspect, the molar ratio of hydroxyl functional groups (—OH) from mono-alcohol (iii) (b) to carboxyl functional groups (—COOH) from poly-carboxylic acid (i) is from about 1:2 to about 1:5. In an aspect, the molar ratio of hydroxyl functional groups (—OH) from mono-alcohol (iii) (b) carboxyl functional groups (—COOH) from poly-carboxylic acid (A) is about 1:16, about 1:15, about 1:14, about 1:13, about 1:12, about 1:11, about 1:10, about 1:9, about 1:8, about 1:7, about 1:6, about 1:5, about 1:4, about 1:3, or about 1:2.

In an aspect, the conversion of carboxyl functional groups (—COOH) to ester functional groups (—CO(O)—) is no less than 80% by mole. The percent conversion is calculated by titration of carboxylic acid functional groups (—COOH) with 0.1N KOH in isopropanol.

In an aspect, esterification is carried out in the absence of a solvent or emollient.

In an aspect, esterification is conducted in solvent or emollient. In another aspect, esterification is conducted in more than one solvent or emollient.

In an aspect, the percentage of solvent or emollient in total raw materials of the esterification reaction is in the range of 0% to 80% by weight. In an aspect, the percentage of solvent or emollient in total raw materials of the esterification reaction is in the range of 0% to 60% by weight. In an aspect, the percentage of solvent or emollient in total raw materials of the esterification reaction is in the range of 0% to 50% by weight. In an aspect, the percentage of solvent or emollient in total raw materials of the esterification reaction is in the range of 0% to 40% by weight. In an aspect, the percentage of solvent or emollient in total raw materials of the esterification reaction is in the range of 0% to 30% by weight. In an aspect, the percentage of solvent or emollient in total raw materials of the esterification is in the range of 0% to 20% by weight. In an aspect, the percentage of solvent or emollient in total raw materials of the esterification reaction is in the range of 0% to 10% by weight. In an aspect, the percentage of solvent or emollient in total raw materials of the esterification reaction is in the range of 10% to 20% by weight. In an aspect, the percentage of solvent or emollient in total raw materials of the esterification reaction is in the range of 20% to 30% by weight.

### 2. Activating Agent

In an aspect, the preparation of the elastomer further comprises an activating agent.

In an aspect, the preparation of the elastomer does not comprise an activating agent.

In an aspect, the activating agent is selected from the group consisting of dimethyl dicarbonate, diethyl dicarbonate, dipropyl dicarbonate, di-tertiary-butyl dicarbonate, and combinations thereof.

### 3. Catalyst

In an aspect, the preparation of the elastomer further comprises a catalyst. In an aspect, the preparation of the elastomer does not comprise a catalyst. However, it has been found that when no catalyst is used the reaction times are protracted.

In an aspect, the catalyst is selected from the group consisting of methanesulfonic acid, p-toluenesulfonic acid, benzene sulfonic acid, sulfuric acid, amidosulfonic acid, sulfamic acid, sodium bisulfate, phosphoric acid, hydrochloric acid, hydrobromic acid, nitric acid, sodium hydroxide, potassium hydroxide, sodium methoxide, sodium ethoxide, potassium methoxide, potassium ethoxide, bismuth (III) neodecanoate, bismuth (III) citrate, bismuth (III) chloride, bismuth (III) acetate, bismuth (III) phosphate, tin chloride, tin-pyrone, dibutyltin dilaurate, di-n butyl-oxo-stannane, butyl stannoic acid, zinc chloride, zinc bromide, zinc carboxylic salt, zinc oxide, zinc hydroxy nitrate salt, zinc hydroxy acetate, triethylamine, tripropylamine, cocamidopropyl dimethylamine, stearamidopropyl dimethylamine, isostearamidopropyl dimethylamine, and combinations thereof. In an aspect, the catalyst is p-toluenesulfonic acid, methanesulfonic acid, phosphoric acid, bismuth neodecanoate, or a combination thereof. In an aspect, the catalyst is methanesulfonic acid.

In an aspect, the catalyst is a salt. In an aspect, the catalyst is a salt selected from the group consisting of Yb(OTf)₃, Sc(OTf)₃, Hf(OTf)₄, Bi(OTf)₃, Al(OTf)₃, Zn(OTf)₂, Mg(ClO₄)₂, Cu(OTf)₂, Ti(OCH(CH₃)₂)₄, and combinations thereof.

### 4. Emollient or Solvent

In an aspect the cosmetically acceptable carrier (C) is preferably selected from the emollients or solvents as described herein below. The cosmetically acceptable carrier (C) can be present in the preparation of the crosslinked polyester component (B) and/or can be added after the preparation of the crosslinked polyester component (B).

In an aspect, the preparation of the polyester elastomer (B) can occur in the presence of a solvent. The solvent can also act as an emollient, preferably a cosmetic emollient. When the solvent acts also as an emollient it also provides a softening, protecting, moisturizing, and/or lubricating effect to the skin. In an aspect, the solvent or emollient is preferably a biobased or naturally derived. In an aspect, the solvent or emollient is a triglyceride solvent, a mono-ester solvent, a di-ester solvent, a citrate ester solvent, an ether solvent, a carbonate solvent, a hydrocarbon solvent, a silicone solvent, or a combination thereof.

In an aspect, the solvent is a triglyceride solvent of formula (XIV)

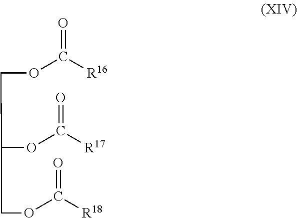

wherein
   each R¹⁶, R¹⁷, and R¹⁸ are independently C1-C35 alkyl group, C1-C35 heteroalkyl group, C2-C35 alkene group, or C2-C35 heteroalkene group.

In an aspect, the solvent is of formula (XIV), wherein R¹⁶, R¹⁷, and R¹⁸ are independently C2-C17 alkyl group or C2-C17 alkylene group.

In an aspect, the solvent is a triglyceride solvent selected from the group consisting of caprylic/capric triglyceride, triheptanoin, corn oil, soybean oil, olive oil, rape seed oil, cotton seed oil, coconut oil, almond oil, argon oil, rosehip oil, black seed oil, grape seed oil, avocado oil, apricot kernel oil, geranium oil, lavender oil, rosehip oil, macadamia oil, eucalyptus oil, sardine oil, herring oil, safflower oil, linseed oil, sunflower oil, olive oil, canola oil, sesame oil, cottonseed oil, palm oil, rapeseed oil, tung oil, fish oil, peanut oil, cuphea oil, milkweed oil, salicornia oil, whale oil, castor oil, and combinations thereof. In an aspect, the triglyceride solvent is selected from the group consisting of caprylic/capric triglyceride, triheptanoin, and combinations thereof.

In an aspect, the solvent is a mono-ester solvent of formula (XV)

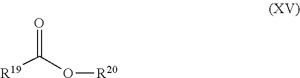

wherein
   R¹⁹ is C1-C35 alkyl group, C1-C35 heteroalkyl group, C2-C35 alkene group, or C2-C35 heteroalkene group; and
   R²⁰ is C1-C35 alkyl group, C1-C35 heteroalkyl group, C2-C35 alkene group, or C2-C35 heteroalkene group.

In an aspect, the solvent is a mono-ester solvent of formula (XV), wherein R¹⁹ is C5-C17 alkyl group or C5-C17 alkene group and R²⁰ is C2-C17 alkyl group or C2-C17 alkene group.

In an aspect, the solvent is a mono-ester solvent selected from the group consisting of coco-caprylate, coco-caprate, jojoba oil, jojoba esters, isopropyl jojobate, ethyl macadamiate, isoamyl laurate, heptyl undecylenate, methylheptyl isostearate, isostearyl isostearate, glyceryl ricinoleate, isostearyl palmitate, myristyl myristate, octyldodecyl myristate, octyldodecyl hydroxystearate, butyl myristate, ethylhexyl cocoate, ethylhexyl palmitate, ethylhexyl stearate, butyl stearate, decyl oleate, isocetyl behenate, isocetyl myristate, isocetyl palmitate, isocetyl stearate, isodecyl oleate, isopropyl isostearate, isopropyl myristate, isopropyl palmitate, oleyl oleate, propylene glycol laurate, octyldodecyl erucate, C12-C13 alkyl lactate, C12-C15 alkyl lactate, isostearyl lactate, glycereth-5-lactate, lauryl lactate, myristyl lactate, oleyl lactate, laureth-2-benzoate, C12-C15 alkyl benzoate, C12-C15 pareth-3-benzoate, dipropylene glycol benzoate, isodecyl salicylate, C12-C15 alkyl salicylate, tridecyl salicylate, ethylhexyl isononanoate, cetyl ethylhexanoate, isononyl isononanoate, isodecyl ethylhexanoate, isodecyl isononanoate, tridecyl ethylhexanoate, isotridecyl isononanoate, isostearyl isononanoate, cetearyl isononanoate, laureth-2-ethylhexanoate, cetearyl ethylhexanoate, isodecyl neopentanoate, isostearyl neopentanoate, nyristyl neopentanoate, isostearyl behenate, octyldodecyl neopentanoate, tridecyl neopentanoate, and combinations thereof. In an aspect, the mono-ester solvent is selected from the group consisting of cococaprylate/caprate, coco-caprylate, jojoba oil, isoamyl laurate, methylheptyl isostearate, C12-C13 alkyl lactate, C12-C15 alkyl lactate, lauryl lactate, ethylhexyl isononanoate, cetyl ethylhexanoate, isononyl isononanoate, isodecyl ethylhexanoate, isodecyl isononanoate, tridecyl ethylhexanoate, isotridecyl isononanoate, isostearyl isononanoate, cetearyl isononanoate, and combinations thereof. In an aspect, the mono-ester solvent is selected from the group consisting of coco-caprylate/caprate, coco-caprylate, isoamyl laurate, isononyl isononanoate, heptyl undecylenate, jojoba oil, jojoba esters, and combinations thereof.

In an aspect, the solvent is:
(a) a di-ester solvent of formula (XVI)

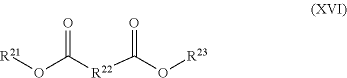

wherein
   R²¹ is C1-C35 alkyl group, C1-C35 heteroalkyl group, C2-C35 alkene group, or C2-C35 heteroalkene group; and
   R²² and R²³ are independently C1-C35 alkyl group, C1-C35 heteroalkyl group, C2-C35 alkene group, or C2-C35 heteroalkene group; or
(b) a di-ester solvent of formula (XVII)

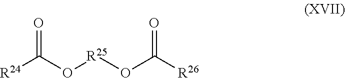

wherein
   R²⁴ is C1-C35 alkyl group, C1-C35 heteroalkyl group, C2-C35 alkene group, or C2-C35 heteroalkene group; and
   R²⁵ and R²⁶ are independently H, C1-C35 alkyl group, C1-C35 heteroalkyl group, C2-C35 alkene group, or C2-C35 heteroalkene group; or
(c) a di-ester solvent of formula (XVIII)

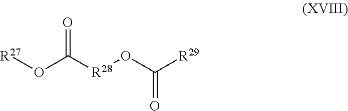

wherein
   R²⁷ and R²⁸ are independently C1-C35 alkyl group, C1-C35 heteroalkyl group, C2-C35 alkene group, or C2-C35 heteroalkene group; and
   R²⁹ is H, C1-C35 alkyl group, C1-C35 heteroalkyl group, C2-C35 alkene group, or C2-C35 heteroalkene group.

In an aspect, the solvent is a di-ester solvent of formula (XVI), formula (XVII), or formula (XVIII), wherein R²³, R²⁶, and R²⁹ is C2-C10 alkyl group or C2-C10 alkene group and R²¹, R²⁴, R²⁷, and R²², R²⁵, and R²⁸ are independently C1-C12 alkyl group or C2-C12 alkene group.

In an aspect, the di-ester solvent is selected from the group consisting of diethyl succinate, dibutyl succinate, diethyhexyl succinate, diisopropyl sebacate, dimethyl sebacate, diethyl sebacate, dibutyl sebacate, diisostearyl dimer, diisostearyl malate, isostearyl stearoyl stearate, isocetyl stearoyl stearate, octyldodecyl stearoyl stearate, diethylhexyl malate, diethylhexyl maleate, dipropylene glycol dibenzoate, dicapryl adipate, dicaprylyl maleate, diisopropyl dimer, diisopropyl adipate, diisobutyl adipate, diisopropyl sebacate, diisostearyl dimer, diethyhexyl succinate, diethylene glycol diethylhexanoate, neopentyl glycol dicaprate, propylene glycol dicaprylate/dicaprate, neopentyl glycol diisostearate, neopentyl glycol diethylhexanoate, neopentyl glycol diheptanoate, and combinations thereof. In an aspect, the di-ester solvent is selected from the group consisting of dicapryl adipate, dicaprylyl maleate, diisopropyl adipate, diisobutyl adipate, diethyl succinate, dibutyl succinate, diethyhexyl succinate, diisopropyl sebacate, dimethyl sebacate, diethyl sebacate, dibutyl sebacate, neopentyl glycol diethylhexanoate, neopentyl glycol diheptanoate, and combinations thereof.

In an aspect, the solvent is a citrate ester solvent of formula (XVIII)

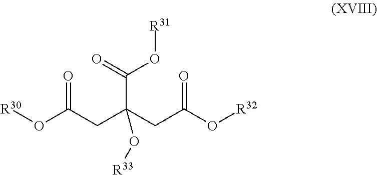

wherein
   R³⁰, R³¹, R³², and R³³ are independently H, C1-C35 alkyl group, C1-C35 heteroalkyl group, C2-C35 alkene group, or C2-C35 heteroalkene group, wherein at least one of R³⁰, R³¹, R³², and R³³ is not H.

In an aspect, the solvent is a citrate ester solvent of formula (XVIII), wherein R³⁰, R³¹, and R³² are independently C1-C10 alkyl group or C2-C10 alkene group and R³³ is an acetyl group.

In an aspect, the solvent is a citrate ester solvent selected from the group consisting of tricaprylyl citrate, triisostearyl citrate, triisocetyl citrate, trioctyldodecyl citrate, triethyl citrate, tributyl citrate, acetyl triethyl citrate, acetyl tributyl citrate, trioctyldodecyl citrate, triisocetyl citrate, and combinations thereof.

In an aspect, the solvent is an ether solvent of formula (XIX)

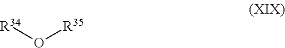

wherein
   R³⁴ and R³⁵ are independently H, C2-C20 alkyl group, C2-C20 heteroalkyl group, C2-C20 alkene group, or C2-C20 heteroalkene group, wherein at least one of R³⁴ and R³⁵ is not H.

In an aspect, the solvent is an ether solvent of formula (XIX), wherein R³⁴ and R³⁵ are independently C2-C20 alkyl group.

In an aspect, the solvent is an ether solvent selected from the group consisting of dicaprylyl ether, didecyl ether, panthenyl ethyl ether, dicetyl ether, dimyristyl ether, distearyl ether, dilauryl ether, and combinations thereof. In an aspect, the ether solvent is selected from the group consisting of dicaprylyl ether, didecyl ether, and combinations thereof.

In an aspect, the solvent is a carbonate solvent of formula (XX)

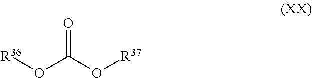

wherein
   R³⁶ and R³⁷ are independently H, C2-C20 alkyl group, C2-C20 heteroalkyl group, C2-C20 alkene group, or C2-C20 heteroalkene group.

In an aspect, the solvent is a carbonate solvent of formula (XX), wherein R³⁶ and R³⁷ are independently C2-C20 alkyl group.

In an aspect, the solvent is a carbonate solvent selected from the group consisting of dicaprylyl carbonate, diethyl hexyl carbonate, and combinations thereof.

In an aspect, the solvent is a hydrocarbon with number of carbon atoms from C4 to C60.

In an aspect, the solvent is a hydrocarbon with a number of carbon atoms from C10 to C50. In an aspect, the solvent is a hydrocarbon with a number of carbon atoms from C20 to C40.

In an aspect, the solvent is a hydrocarbon solvent selected from the group consisting of farnesene, hydrogenated farnesene, coconut alkanes, coconut/palm kernel alkanes, C9-C12 alkane, C10-C13 alkane, C12-C17 alkane, C13-C14 alkane, C13-C15 alkane, C14-C17 alkane, C14-C19 alkane, C14-C20 alkane, C14-C22 alkane, C15-C19 alkane, C21-C28 alkane, C17-C23 alkane, C9-C12 isoalkane, C9-C13 isoalkane, C9-C14 isoalkane, C9-C16 isoalkane, C10-C11 isoalkane, C10-C12 isoalkane, C10-C13 isoalkane, C11-C12 isoalkane, C11-C13 isoalkane, C11-C14 isoalkane, C12-C14 isoalkane, C12-C15 isoalkane, C12-C20 isoalkane, C13-C14 isoalkane, C13-C16 isoalkane, C14-C16 isoalkane, C15-C19 isoalkane, C10-C16 olefin, C12-C18 olefin, C18-C26 olefin, C20 olefin, C20-C24 olefin, C24-C30 olefin, C26-C28 olefin, C26-C54 olefin, C28-C36 olefin, C28-C52 olefin, C30-C38 olefin, C30-C45 olefin, C4-C12 olefin, C4-C6 olefin, C5-C6 olefin, hydrogenated poly(C6/C10/C14 olefin), hydrogenated poly(C6-C12 olefin), hydrogenated poly(C6-C14 olefin), hydrogenated poly(C6-C20 olefin), hydrogenated poly(C8/C12 olefin), poly(C20-C28 olefin), poly(C30-C45 olefin), poly(C4-C12 olefin), poly(C6-C14 olefin), hexadecene, C32 alkane, C32 isoalkane, C54 alkane, C54 isoalkane, diethylhexylcyclohexane, undecane, tridecane, tetradecane, pentadecane, hexadecane, octadecane, docosane, squalane, hydrogenated polyisobutene, polybutene, hydrogenated polydecene, hydrogenated didecene, mineral oil, liquidum, petrolatum, dodecane, isohexadecane, isododecane, isoeicosane, and combinations thereof. In an aspect, the hydrocarbon solvent is selected from the group consisting of squalane, farnesene, hydrogenated farnesene, coconut alkanes, C9-C12 alkane, C13-C15 alkane, C14-C19 alkane, C14-C20 alkane, C14-C22 alkane, C15-C19 alkane, C13-C16 isoalkane, dodecane, undecane, tridecane, tetradecane, pentadecane, hexadecane, hexadecene, octadecane, squalane, isododecane, isohexadecane, C32 alkane, C32 isoalkane, C54 alkane, C54 isoalkane, and combinations thereof. In an aspect, the hydrocarbon solvent is selected from the group consisting of squalane, hydrogenated farnesene, coconut alkanes, C9-C12 alkane, C13-C15 alkane, C13-C16 isoalkane, C14-C19 alkane, dodecane, tetradecane, isododecane, hexadecane, octadecane, hexadecene, C32 alkane, C32 isoalkane, C54 alkane, C54 isoalkane, and combinations thereof.

In an aspect, the hydrocarbon solvent is selected from the group consisting of squalane, C32 alkane, C32 isoalkane, C54 alkane, C54 isoalkane, and combinations thereof.

In an aspect, the solvent may be a chemical UV filter. The UV filter solvent may be selected from the group consisting of ethyl dimethyl PABA, ethylhexyl methoxycinnamate, ethylhexyl salicylate, homosalate, isoamyl p-Methoxycinnamate, menthyl anthranilate, octocrylene, polysilicone-15, terepthalyidene dicamphor sulfonic acid, triethanolamine salicylate, and combinations thereof.

In an aspect, the solvent is a silicone solvent selected from the group consisting of dimethicone, phenyl dimethicone, caprylyl methicone, ethyl trisiloxane, cyclotetrasiloxane, cyclopentasiloxane, cyclohexasiloxane, and combinations thereof.

In an aspect, a defined amount of solvent is used in the preparation of the polyester elastomer. In an aspect, the amount of solvent is from 0% to 70% of the total weight of poly-carboxylic acid (i), polyol (ii), mono-carboxylic acid (iii) (a) and mono-alcohol (iii) (b). In an aspect, the amount of solvent is from 0% to 50% of the total weight of poly-carboxylic acid (i), polyol (ii), mono-carboxylic acid (iii) (a) and mono-alcohol (iii) (b). In an aspect, the amount of solvent is from 0% to 40% of the total weight of poly-carboxylic acid (i), polyol (ii), mono-carboxylic acid (iii) (a) and mono-alcohol (iii) (b). In an aspect, the amount of solvent is from 0% to 30% of the total weight poly-carboxylic acid (i), polyol (ii), mono-carboxylic acid (iii) (a) and mono-alcohol (iii) (b). In an aspect, the range of solvent is from 0% to 20% of the total weight of poly-carboxylic acid (i), polyol (ii), mono-carboxylic acid (iii) (a) and mono-alcohol (iii) (b). In an aspect, the amount of solvent is from 10% to 50% of the total weight of poly-carboxylic acid (i), polyol (ii), mono-carboxylic acid (iii) (a) and mono-alcohol (iii) (b). In an aspect, the amount solvent is 50%, 40%, 30%, 20%, or 10% of the total weight of poly-carboxylic acid (i), polyol (ii), mono-carboxylic acid (iii) (a) and mono-alcohol (iii) (b).

In an aspect, the amount of solvent is from 0% to 30% of the total weight of poly-carboxylic acid (i) and mono-carboxylic acid (iii) (a). In an aspect, the amount of solvent is from 0% to 20% of the total weight of poly-carboxylic acid (i) and mono-carboxylic acid (iii) (a). In an aspect, the amount of solvent is from 0% to 10% of the total weight of poly-carboxylic acid (i) and mono-carboxylic acid (iii) (a). In an aspect, the amount of solvent is from 10% to 30% of the total weight of poly-carboxylic acid (i) and mono-carboxylic acid (iii) (a). In an aspect, the solvent is 30%, 20%, or 10% of the total weight of poly-carboxylic acid (i) and mono-carboxylic acid (iii) (a).

In an aspect, no solvent is used to prepare the elastomer (B).

In an aspect, a solvent is used to prepare the polyester elastomer (B), which is removed after preparing the polyester elastomer (B) to form a polyester elastomer (B) powder. In an aspect, to the polyester elastomer powder a solvent/emollient can be added again to form a polyester elastomer (B) gel, optionally applying a shear force as described below.

In an aspect, polyester elastomer (B) is made from C36 dimer acid, diglycerol, and isostearic acid with about 10% to about 40% by weight squalane based on the total weight of the polyester elastomer (B) and the squalane. That is, in an aspect the invention relates to a composition of a polyester elastomer made from C36 dimer acid, diglycerol, isostearic acid, and squalane, comprising about 10% to about 40% by weight squalane.

In an aspect, polyester elastomer (B) is made from hydrogenated C36 dimer acid, diglycerol, and oleic acid without any solvent or emollient.

### 5. Temperature

In an aspect, the method of preparing the elastomer comprises reacting at least one poly-carboxylic acid, poly-carboxylic acid ester, or combination thereof, at least one polyol, and/or at least one mono-carboxylic acid, and/or at least one mono-alcohol with mixing at a pre-determined temperature until an elastomer is formed. In an aspect, the method of preparing the elastomer comprises reacting at least one poly-carboxylic acid, poly-carboxylic acid ester, or combination thereof, at least one polyol, and/or at least one mono-carboxylic acid, and/or at least one mono-alcohol, optionally at least one solvent or emollient, and optionally a catalyst with mixing at a pre-determined temperature until an elastomer is formed. In an aspect, the temperature range is from 30° C. to 250° C.

In an aspect, the reaction occurs at a temperature from about 30° C. to about 250° C. In an aspect, the reaction occurs at a temperature from about 60° C. to about 250° C. In an aspect, the reaction occurs at a temperature from about 30° C. to about 125° C. or about 40° C. to about 100° C. In an aspect, the reaction occurs at a temperature of about 30° C., about 35° C., about 40° C., about 45° C., about 50° C., about 55° C., about 60° C., about 65° C., about 70° C., about 75° C., about 80° C., about 85° C., about 90° C., about 95° C., about 100° C., about 105° C., about 110° C., about 115° C., about 120° C., about 125° C., about 130° C., about 135° C., about 140° C., about 145° C., about 150° C., about 155° C., about 160° C., about 165° C., about 170° C., about 175° C., about 180° C., about 185° C., about 190° C., about 195° C., about 200° C., about 205° C., about 210° C., about 215° C., about 220° C., about 225° C., about 230° C., about 235° C., about 240° C., about 245° C., or about 250° C.

### 6. Time

In an aspect, the reaction time is from about 12 hours to about 150 hours. In an aspect, the reaction time is from about 6 hours to about 24 hours. In an aspect, the reaction time is from about 8 hours to about 27 hours. In an aspect, the reaction time is about 6 hours, about 6.5 hours, about 7 hours, about 7.5 hours, about 8 hours, about 8.5 hours, about 9 hours, about 9.5 hours, about 10 hours, about 10.5 hours, about 11 hours, about 11.5 hours, about 12 hours, about 12.5 hours, about 13 hours, about 13.5 hours, about 14 hours, about 14.5 hours, about 15 hours, about 15.5 hours, about 16 hours, about 16.5 hours, about 17 hours, about 17.5 hours, about 18 hours, about 18.5 hours, about 19 hours, about 19.5 hours, about 20 hours, about 20.5 hours, about 21 hours, about 21.5 hours, about 22 hours, about 22.5 hours, about 23 hours, about 23.5 hours, about 24 hours, about 24.5 hours, about 25 hours, about 25.5 hours, about 26 hours, about 26.5 hours, about 27 hours, about 28 hours, about 29 hours, about 30 hours, about 31 hours, about 32 hours, about 33 hours, about 34 hours, about 35 hours, about 36 hours, about 37 hours, about 38 hours, about 39 hours, about 40 hours, about 41 hours, about 42 hours, about 43 hours, about 44 hours, about 45 hours, about 46 hours, about 47 hours, about 48 hours, about 49 hours, about 50 hours, about 55 hours, about 60 hours, about 65 hours, about 70 hours, about 75 hours, about 80 hours, about 85 hours, about 90 hours, about 95 hours, about 100 hours, about 105 hours, about 110 hours, about 115 hours, about 120 hours, about 125 hours, about 130 hours, about 135 hours, about 140 hours, about 145 hours, or about 150 hours.

The reaction time can be adjusted by determination of the gel fraction achieved, and preferably the reaction time is such that the gel fraction of the polyester elastomer is greater than 60%. A method to measure the gel fraction in the polyester elastomer is described below.

### 7. By-Product Removal

In an aspect, the method further comprises removing water and alcohol by-product from the reaction. In a further aspect, the water and alcohol by-products are removed from the reaction by mixing and heating the reaction. In an aspect, the reaction is heated to above about 120° C. to remove the water and alcohol by-products. In an aspect, the water and alcohol by-products are removed from the reaction by nitrogen flow, by vacuum, or a combination thereof. In an aspect, water is removed by nitrogen stripping and vacuum, which have an impact on the reaction time.

As mentioned above in an aspect, the polyester elastomer (B) is provided as a (at room temperature (23° C.) flowable or fluid) gel or a (dry) powder.

In an aspect, the polyester elastomer (B) is processed into a gel as described herein.

In an aspect, polyester elastomer (B) used is only comprised of crosslinked polyester without solvent or emollient. In an aspect, polyester elastomer (B) used is comprised of polyester without solvent or emollient. In an aspect, polyester elastomer (B) used is comprised of crosslinked polyester with solvent or emollient. In an aspect, polyester elastomer (B) used is comprised of polyester with solvent or emollient.

Solvents or emollients that can be used to prepare the polyester elastomer composition (B) are as described herein above and can be selected from the solvents or emollients as defined herein. In another aspect, the polyester elastomer in polyester elastomer composition (B) used is in the range of 5 wt % to 100 wt %. In another aspect, the polyester elastomer in polyester elastomer composition (B) used is in the range of 5 wt % to 70 wt %. In another aspect, the polyester elastomer in polyester elastomer composition (B) used is in the range of 10 wt % to 60 wt %. In another aspect, the polyester elastomer in polyester elastomer composition (B) used is in the range of 20 wt % to 50 wt %.

In another aspect, the crosslinked polyester in polyester elastomer composition (B) used is in the range of 5 wt % to 50 wt %. In another aspect, the crosslinked polyester in polyester elastomer composition (B) used is in the range of 5 wt % to 30 wt %. In another aspect, the crosslinked polyester in polyester elastomer composition (B) used is in the range of 10 wt % to 30 wt %. Solvents or emollients that can be used to prepare the polyester elastomer composition are described herein and can be selected from the solvents or emollients as defined herein.

In an aspect, the polyester elastomer composition (B) used comprises at least one solvent or emollient added to the polyester elastomer during the shearing force process to form a gel. Solvents or emollients that can be used to prepare the polyester elastomer composition are described herein and can be selected from the solvents as defined herein. In an aspect, the solvent or emollient is from about 20% to about 95% weight by weight of the composition. In an aspect, the solvent or emollient is from about 20% to about 50% weight by weight of the composition. In an aspect, the solvent or emollient is from about 50% to about 90% weight by weight of the composition. In an aspect, the solvent or emollient is from about 70% to about 90% weight by weight of the composition. In an aspect, the polyester elastomer composition (B) comprises from about 50% to about 90% weight by weight of solvent or emollient, from about 50% to about 80% weight by weight of solvent or emollient, from about 50% to about 70% weight by weight of solvent or emollient, or from about 50% to about 60% weight by weight of solvent or emollient. In some embodiments, the polyester elastomer composition (B) comprises about 20%, about 30%, about 40%, about 50%, about 60%, about 70%, about 80%, or about 90% weight by weight of solvent or emollient.

Process to prepare the polyester elastomer component (B) as a gel composition

In an aspect, the polyester elastomer obtained by the esterification process is crumbled or processed to form a polyester elastomer powder. In another aspect, the polyester elastomer is processed by a three-roll mill to form a polyester elastomer powder. The polyester elastomer composition (B) can be also obtained as a powder if it still comprises a solvent or emollient added to the reaction mixture.

In an aspect, polyester elastomer composition (B) is prepared by combining the polyester elastomer with one or more solvents or emollients to form a polyester elastomer gel.

In an aspect, polyester elastomer and solvent/emollient mixture are processed with a homogenizer to produce a gel optionally applying a shear force, e.g. by a high-shear disperser mixer.

In an aspect to prepare the polyester elastomer composition (B) as gel, a polyester elastomer is swelled in a solvent or emollient before being processed to make a gel at a temperature under 23° C. In an aspect, the time of polyester elastomer swelling in a solvent or emollient is from 1 hour to 1 week. In an aspect, the elastomer is subject to swelling in a solvent or emollient from 10 minutes to 1 week, from 10 minutes to 4 days, from 10 minutes to 3 days, from 10 minutes to 2 days, from 10 minutes to 1 day, from 10 minutes to 12 hours, from 10 minutes to 6 hours, from 10 minutes to 3 hours, from 10 minutes to 2 hours, from 10 minutes to 1 hour, or from 10 minutes to 30 minutes.

Once the initially produced polyester elastomer is prepared, it can be mixed with an additional quantity of at least one solvent or emollient that can be different from the solvent emollient used to prepare the initially produced elastomer. In some aspects, the at least one solvent or emollient used to prepare the elastomer is the same as the at least one solvent or emollient used to prepare the elastomer composition. The addition of an additional quantity of at least one solvent or emollient dilutes the gel composition and thereby adjusts its viscosity.

The method of preparing the polyester elastomer composition (B) as gel, suitably comprises:
(i) combining a polyester elastomer with at least one solvent or emollient thereby forming a swollen polymer elastomer; and
(ii) subjecting the swollen polyester elastomer to shear force thereby forming a polyester elastomer composition.

In an aspect, the polyester elastomer composition is a powder, a gel, or a paste.

In an aspect, the at least one solvent or emollient is selected from the solvents or emollients described herein.

### Synthesis of the Polyester Elastomer Composition (B) in Biobased or Naturally Derived Solvent

Polyester elastomer (B) can be synthesized in a biobased or naturally derived solvent, which requires a reaction vessel that is equipped with Nitrogen flow, heating capacity, high viscosity mixing, and the ability to distill off water.

For example, to the vessel, 1.1 equivalents of dimer acid are added, along with 1 equivalent of diglycerol and 0.75 equivalents of isostearic acid. The raw materials are followed by the addition of 10-70 wt % of a biobased or naturally derived solvent, which may be a triglyceride solvent, a mono-ester solvent, a di-ester solvent, a citrate ester solvent, an ether solvent, a carbonate solvent, a hydrocarbon solvent, a silicone solvent, and combinations thereof. After all ingredients are charged under agitation, the temperature of the mass is raised to 160° C., and water is stripped off as it forms. The temperature is held until gelation takes place and the polymer elastomer rubber is formed, which occurs between 20 and 150 hours. Thereafter, the elastomer is broken into a powder by mechanical stirring. This elastomer rubber powder is then characterized by its swell value and % crosspolymer value, which are determined by the swell test and Soxhlet extraction, described below. On average, the natural elastomer rubber is composed of 65-95% crosslinked polymer.

### Characterization of the Crosslinked Polyester Components (B)

In an aspect, the solubility of polyester elastomer (B) is measured by mixing 1 gram of polyester elastomer with 100 gram of test solvent with magnetic stirring in a sealed glass container for 24 hours under room temperature (23° C.). Afterward the polyester elastomer (B) and test solvent is passed through filtration and the solid on filter is obtained and dried under 80° C. for 20 hours or dried to constant weight, optionally in vacuo. The dried solid is considered as the fraction of polyester elastomer (B) which is not soluble in the test solvent.

In an aspect, the fraction of the polyester elastomer (B) which is not soluble in the ethyl acetate by weight is greater than or equal to 40% of the total weight of the polyester elastomer (B).

In another aspect, the fraction of the polyester elastomer (B) which is not soluble in ethyl acetate is 40% to 90% of the total weight of the polyester elastomer (B). In another aspect, the fraction of the polyester elastomer (B) which is not soluble in ethyl acetate is 50% to 90% of the total weight of the polyester elastomer (B).

A sample of polyester elastomer (B) without solvent or emollient, or a sample of polyester elastomer (B) with solvent or emollient can be used. In case a sample of polyester elastomer (B) with solvent or emollient is used, the weight of the solvent or emollient in the composition is considered and subtracted from the total composition weight to determine the weight percentage of the fraction of the polyester elastomer (B) which is not soluble in the ethyl acetate.

The method of Soxhlet extraction can be used to determine the % by weight of crosslinked polyester (gel fraction) in the polyester elastomer (B). The basis for the test is to extract soluble low Mw components (and optionally present solvents or emollients) from the high Mw and crosslinked insoluble components through an extraction test. The percentage of the crosslinked polyester is defined as the ratio of the weight of insoluble residue (dried gel) to the initial weight of the polyester elastomer sample.

The gel fraction of the polyester elastomers (B), defined as

$$Gel fraction ( \% ) = 100 \times \frac{weight of dried gel ( insoluble residue of the extraction )}{total weiht of the polyester elastomer used in the extraction}$$

is determined as follows.

A sample of polyester elastomer without solvent or emollient, or a sample of polyester elastomer with solvent or emollient can be used. In case a sample of polyester elastomer with solvent or emollient is used, the weight of the solvent or emollient in the polyester elastomer is considered and subtracted from the sample weight to determine the total weight of the polyester elastomer (B) used in the extraction in the formula above.

A cellulose extraction thimble is weighed. 3.0-3.5 grams of a sample of a polyester elastomer is weighed and placed in the cellulose extraction thimble. About 125-150 mL of ethyl acetate (EtOAc) are placed in a 250 mL round bottom flask. The thimble with the sample is placed into the Soxhlet extraction column. The EtOAc solution is heated to 80° C. (Reflux, bp 77° C.) and maintained at reflux for 1 hour to extract the polymer sample solubles and the optional present solvent or emollient. After 2 hours the EtOAc solution is allowed to cool to room temperature (about 23° C.). The thimble containing the polymer residue is removed from the Soxhlet apparatus. The thimble is placed in a desiccator under vacuum, a vacuum oven, or a vented oven (50° C.) to remove the residual EtOAc (24 hours). After the EtOAc has been removed, the weight of the thimble containing the dried gel is determined. The weight of the dried gel is calculated by subtraction of the weight of the cellulose extraction thimble. The gel fraction is calculated from the above equation. Any amount of solvent or emollient in the sample is subtracted from the “total weight of the polyester elastomer (B) used in the extraction”.

In an aspect, the gel fraction of the polyester elastomer (B) is greater than 20%. In an aspect, the gel fraction of the polyester elastomer (B) is greater than 40%. In an aspect, the gel fraction of the polyester elastomer (B) is greater than 50%. In an aspect, the gel fraction of the polyester elastomer is greater than 60%. In an aspect, the gel fraction of the polyester elastomer (B) is greater than 70%.

Swelling test for the polyester elastomer (B) is to determine the swelling capacity of the polyester elastomer (B) through a weight of solvent or emollient retained by the polyester elastomer. The swelling ratio (SR-sometimes called swelling value) is determined according to the equation:

$$SR = \frac{Ws - Wi}{Wi}$$

where:
   Ws is the weight of the swollen polyester elastomer, and
   Wi is the weight of the initial (dry polymer).

The swelling ratio of the polyester elastomer (B) is suitably determined as follows.

A sample of polyester elastomer (B) without solvent or emollient, or a sample of polyester elastomer with solvent or emollient can be used. In case a sample of polyester elastomer (B) with solvent or emollient is used, the weight of the solvent or emollient already present in the composition is considered and subtracted from the initial sample weight Wi.

The swelling procedure is carried out at ambient temperature (23° C.).

About 1.9-2.1 grams of the polyester elastomer is placed in a 25 mL beaker. In the same beaker the polyester elastomer is mixed with 24.9-25.1 grams of coco-caprylate/caprate as solvent. The polyester elastomer is allowed to disperse and absorb (swell) the solvent for 30 minutes. The weight of the filter component (such as Thermo Scientific™ Nalgene™ Rapid-Flow™ Sterile Disposable Filter Units) is determined. After the polyester elastomer has swelled, the mixture in the beaker is mixed and poured into the filter. The beaker is rinsed with about 4.9-5.1 grams of coco-caprylate/caprate solvent to complete transfer of the swelled polyester elastomer. The excess solvent in the gel mixture is allowed to pass through the filter. The filter with swollen polyester elastomer is weighed when no excess solvent is observed on its surface (which can take about 4-18 hours) to give Ws.

The swelling ratio (SR) is calculated by the equation above.

In an aspect, the swelling ratio of the elastomer (B) is from about 1 gram/gram to about 15 gram/gram, from about 1 gram/gram to about 5 gram/gram, from about 1 gram/gram to about 4 gram/gram, from about 1 gram/gram to about 2 gram/gram. In some embodiments, the swelling value of the elastomer is about 15 gram/gram, about 14 gram/gram, about 13 gram/gram, about 12 gram/gram, about 11 gram/gram, about 10 gram/gram, about 9 gram/gram, about 8 gram/gram, about 7 gram/gram, about 6 gram/gram, 5 gram/gram, about 4.8 gram/gram, about 4.6 gram/gram, about 4.4 gram/gram, about 4.2 gram/gram, about 4 gram/gram, about 3.8 gram/gram, about 3.6 gram/gram, about 3.4 gram/gram, about 3.2 gram/gram, about 3 gram/gram, about 2 gram/gram, or about 1 gram/gram.

Polyester Elastomer Component (B) used as a Gel

As mentioned before in an aspect, a polyester elastomer composition (B) is prepared by shearing the polyester elastomer with a solvent or emollient, as described herein, to form a sheared polyester elastomer gel. In another aspect, a polyester elastomer gel is prepared by combining the polyester elastomer, as described herein, with a solvent or emollient, as described herein, thereby forming a mixture and shearing the mixture.

In an aspect, the shear force is provided by any type of mixing and shearing equipment. In an aspect, the mixing and shearing equipment is batch mixer, planetary mixer, single or multiple screw extruder, dynamic or static mixer, colloid mill, homogenizer, sonolator, three roll mill, or a combination thereof.

Subjecting these polyester elastomer compositions to a shearing force produces a polyester elastomer gel (B) which is particular suitable for use in the personal care or cosmetic applications that has an improved spreadability and an improved substance or feel. The claimed cosmetic or personal care applications where this property is most desirable included, but is not limited to, can have for example the form of skin creams, facial creams, hair care products such as shampoos, mousses, and styling gels, protective creams, color cosmetics such as lipsticks, foundations, blushes, makeup, and mascara, and other cosmetic formulations, each comprising skin care active compounds (A).

In an aspect, the viscosity of the polyester elastomer (B) used in gel form is from about 10 cp to about 1,000,000 cp as measured by rheometer at a shear rate of 0.1 s⁻¹. In an aspect, the viscosity of the gel at 25° C. is from about 30,000 cp to about 900,000 cp. In an aspect, the viscosity of the gel is about 10 cp, about 1,000 cp, about 5,000 cp, about 10,000 cp, about 15,000 cp, about 20,000 cp, about 25,000 cp, about 30,000 cp, about 35,000 cp, about 40,000 cp, about 45,000 cp, about 50,000 cp, about 55,000 cp, about 60,000 cp, about 65,000 cp, about 70,000 cp, about 75,000 cp, about 80,000 cp, about 85,000 cp, about 90,000 cp, about 95,000 cp, about 100,000 cp, about 150,000 cp, about 200,000 cp, about 250,000 cp, about 300,000 cp, about 350,000 cp, about 400,000 cp, about 450,000 cp, —about 500,000 cp, about 550,000 cp, about 600,000 cp, about 650,000 cp, about 700,000 cp, about 750,000 cp, about 800,000 cp, about 850,000 cp, about 900,000 cp, about 950,000 cp, or about 1,000,000 cp.

The viscosity of the polyester elastomer gel is measured by Anton Paar rheometer MCR 301, with probe PP25/S at gap 1 mm. The measuring profile is to use flow curve with shear rate 0.01-100/s under temperature 25° C. In measurement, a sample is loaded onto the rheometer stage, the probe is lowered, and the sample is allowed to equilibrate for 3 minutes, after which the test is performed. The viscosity at 10/s is reported.

In an aspect, the polyester elastomer (B) used as gel is comprised of particles of size from about 1 μm to about 500 μm as measured by a laser diffraction particle size analyzer. In an aspect, the gel is comprised of particles of size from about 20 μm to about 400 μm. In an aspect, the gel is comprised of particles of size of about 1 μm, about 5 μm, about 10 μm, about 15 μm, about 20 μm, about 25 μm, about 30 μm, about 35 μm, about 40 μm, about 45 μm, about 50 μm, about 75 μm, about 100 μm, about 125 μm, about 150 μm, about 175 μm, about 200 μm, about 225 μm, about 250 μm, about 275 μm, about 300 μm, about 325 μm, about 350 μm, about 375 μm, or about 400 μm.

In an aspect, the polyester elastomer (B) used as gel is comprised of particles of size with D10 between 10 μm to 50 μm, D50 between 20 μm to 100 μm, and D90 between 30 μm to 200 μm.

The size of particles in polyester elastomer (B) used as gel is measured by HORIBA Scientific Partica LA-960 Laser Scattering Particle Size Analyzer. The sample is prepared by blending 0.3 g of elastomer gel (B) with 15 g of coco-caprylate/caprate and mixing thoroughly at 23° C. A few drops of the diluted gel sample are transferred into the cuvette containing neat coco-caprylate/caprate while constantly stirring. Once the transmittance reaches the acceptable range, the measurement is performed at 23° C. Particle size values of D10, D50 and D90 are reported. The parameter D10 signifies the point in the size distribution, up to and including which, 10% of the total volume of material in the sample is ‘contained’. The parameter D50 signifies the point in the size distribution, up to and including which, 50% of the total volume of material in the sample is ‘contained’. The parameter D90 signifies the point in the size distribution, up to and including which, 90% of the total volume of material in the sample is ‘contained’.

Polyester elastomer (B) as a gel according to the present invention are characterized by the oscillation amplitude as well as oscillation frequency-dependent rheology tests at 25° C. In the linear viscoelastic region within the frequency range from 0.01-100 Hz, a gel has a storage modulus G′ which is always greater than the loss modulus G″. G′ and G″ here are rheological parameters known to the person skilled in the art. The elastic or storage modulus, designated as G′, is an indicator of how elastic the material is i.e., how much mechanical energy is being stored per cycle of deformation whereas, the viscous or loss modulus, namely G″, is the measure of the lost or dissipated mechanical energy as heat and/or other form per cycle of deformation and they collectively quantify the elastic or viscous fraction of viscoelastic solids and/or liquids and are described for example in Ferry, J. D., Viscoelastic Properties of Polymers, John Wiley & Sons, Inc. New York, 1980.

The polyester elastomer (B) as gels used according to the invention have an excellent yield point which, for example, has an advantageous effect on their thickening properties and also their ability to stabilize dispersed constituents of personal care formulations. For dynamic oscillation rheology tests, for example a MCR 301 Rheometer (Anton Paar, Graz, Austria) equipped with a 25 mm parallel plate steel geometry can be used.

Polyester elastomer (B) used as gels are notable for the fact that, at a shear rate of 1 1/s and a temperature of 25° C., they have a viscosity of less than 100,000,000 cp and at the same time satisfies G′>G″; Tan-δ<1 within the linear viscoelastic region demonstrating a frequency nearly invariant characteristics. The polyester gels prepared by the methods described herein are characterized by good flowability, which has an advantageous effect on their handleability and processability, but nevertheless have a pronounced yield point and therefore good thickening and stabilizing properties.

In an aspect, the storage modulus (G′) of the polyester elastomer (B) used as gel is from about 10 Pa to about 100,000 Pa as measured by rheometer within linear viscoelastic region using dynamic-42-rheology. In an aspect, the storage modulus (G′) of the gel is from about 100 Pa to about 50,000 Pa. In an aspect, the storage modulus (G′) of the gel is from about 500 Pa to about 30,000 Pa, In an aspect, the storage modulus (G′) of the gel is about 10 Pa, about 100 Pa, about 500 Pa, about 700 Pa, about 800 Pa, about 1,000 Pa, about 1,500 Pa, about 2,000 Pa, about 2,500 Pa, about 5,000 Pa, about 10,000 Pa, about 15,000 Pa, about 25,000 Pa, about 50,000 Pa, or about 100,000 Pa.

In an aspect, the loss modulus (G″) of the polyester elastomer (B) used as gel is from about 10 Pa to about 100,000 Pa as measured by rheometer within linear viscoelastic region using dynamic rheology. In an aspect, the loss modulus (G″) of the gel is from about 100 Pa to about 50,000 Pa. In an aspect, the loss modulus (G″) of the gel is from about 500 Pa to about 30,000 Pa, In an aspect, the loss modulus (G″) of the gel is about 10 Pa, about 100 Pa, about 500 Pa, about 700 Pa, about 800 Pa, about 1,000 Pa, about 1,500 Pa, about 2,000 Pa, about 2,500 Pa, about 5,000 Pa, about 10,000 Pa, about 15,000 Pa, about 25,000 Pa, about 50,000 Pa, or about 100,000 Pa.

The storage modulus G′ and loss modulus G″ of the polyester elastomer (B) used as gel are measured by Anton Paar rheometer MCR 301, with probe PP25/S at gap 1 mm. The measuring profile is to use flow curve with shear rate 0.01-100/s under temperature 25° C. The measuring profile is to use amplitude sweep with oscillatory strain 0.001-100%, with frequency 1 Hz and under temperature 25° C. In measurement sample is loaded onto the rheometer stage, the probe is lowered and the sample is allowed to equilibrate for 3 minutes after which the amplitude sweep test is performed. The LVR region is determined and the respective G′ value is reported.

In an aspect, the polyester elastomer composition is prepared using the methods described herein.

In an aspect, the polyester elastomers described herein are produced using the principles of green chemistry. In an aspect, the polyester elastomers described herein are produced by a simple, efficient environmentally friendly process, with no toxic raw materials used, and no toxic side products generated.

In an aspect, the polyester elastomer gels described herein are produced using the principles of green chemistry.

In an aspect, the polyester elastomer gels described herein are produced by a simple, efficient environmentally friendly process, with no toxic raw materials used, and no toxic side products generated.

### Cosmetic or Personal Care Formulations

The present disclosure relates to cosmetic, i.e. personal care formulations comprising at least one skin care compound (A), at least one crosslinked polyester component (B), optionally at least one cosmetically acceptable carrier (C) each as described above, and optionally one or more other components commonly used in the cosmetic field (D). Components (A), (B), (C) and (D) of the composition are each different from each other.

In some aspects, the other components commonly used in the cosmetic field (D) are selected from the group consisting of pigment, conditioner, oil, suspending agent, surfactant, emulsifier, preservative, rheology modifier, pH adjustor, reducing agent, anti-oxidant, foaming agents, de-foaming agents, chelating agents, gums or thickeners, oils, waxes, a fragrances, essential oils, and combinations thereof.

In an aspect, the personal care formulation can be a personal care application, such as a deodorant, an antiperspirant, a skin cream, a facial cream, a hair shampoo, a hair conditioner, a mousse, a hair styling gel, a hair spray, a protective cream, a lipstick, a facial foundations, lushes, makeup, and mascara, a skin care lotion, a moisturizer, a facial treatment, a personal cleanser, a facial cleanser, a bath oil, a perfume, a shaving cream, a pre-shave lotion, an after-shave lotion, a cologne, a sachet, or a sunscreen.

The cosmetic compositions comprising the one or more skin care active compounds (A) are in particular whitening ingredients such as ascorbyl palmitate, kojic acid, lucinol, and oil-soluble licorice extract, wrinkle preventers such as retinol, retinoic acid, retinol acetate and retinol palmitate, circulation improving ingredients such as capsaicin and vanillylamide nonylate, antimicrobial ingredients such as isopropylmethylphenol and vitamins such as Vitamin C, vitamin D and Vitamin K as described herein.

The stability and (controlled) delivery of the skin care active compounds (A) afforded by a cosmetic composition is directly related to the amount and type of skin care active compounds (A) contained therein. Particularly, cosmetic compositions must provide good stability upon storage and controlled release of the skin care active compounds (A) to the skin, at the site of application while maintaining a satisfactory sensory perception, such as a smooth and dry touch but not greasy feel upon application.

Table 1 shows some specific examples of the skin care active compounds (A) used in the cosmetic compositions of the invention.

| Chemical Name | INCI Name |
|---|---|
| Aloe Vera | Aloe Barbadensis Leaf Extract |
| Green Tea Extract | Camellia Sinensis Leaf Extract |
| Chamomile Extract | Chamomilla Recutita (Matricaria) Flower Extract |
| Rosehip Oil | Rosa Canina Fruit Oil |
| Turmeric Extract | Curcuma Longa (Turmeric) Root Extract |
| Centella Asiatica (Gotu Kola) Extract | Centella Asiatica Extract |
| Licorice Root Extract | Glycyrrhiza Glabra (Licorice) Root Extract |
| Cucumber Extract | Cucumis Sativus (Cucumber) Fruit Extract |
| Shea Butter | Butyrospermum Parkii (Shea) Butter |
| Calendula Extract | Calendula Officinalis Flower Extract |
| Grape seed extract | Vitis Vinifera (Grape) Seed Extract |
| Sea Buckthorn Oil | Hippophae Rhamnoides Oil |
| Willow Bark Extract | Salix Alba (Willow) Bark Extract |
| Ginseng Extract | Panax Ginseng Root Extract: |
| Green Rooibos Tea | Aspaalthus linearis Leaf Extract |
| Vitamin A | Retinol/Retinyl Palmitate/vitamin A |
| Vitamin B-3 | Niacinamide |
| Vitamin C & derivatives | Ascorbic Acid |
| Coenzyme Q10 | Coenzyme Q10 |
| Polyphenols | Polyphenols |
| Stilbenes | Resveratrol and derivatives thereof |
| Piceatannol | Astringenin |
| Olive oil | Olea Europaea Fruit Oil |
| Almond Oil | Prunus amygdalus dulcis Oil |
| Trehalose | Trehalose |
| Oxyreserveratrol | Oxyreserveratrol |
| Beta Glucan | Beta Glucan |
| Azelaic Acid | Azelaic Acid |
| THD | Tetrahexyldecyl Ascorbate |
| MEP | METHYL ESTRADIOLPROPANOATE (MEP) |
| Superoxide Dismutase | Superoxide Dismutase |
| Elastin | Elastin |
| Fibroblast | Fibroblast |
| Copper peptide | Copper Peptide |
| Tranexamic acid | Tranexamic Acid |
| DMAE & its derivatives | Dimethylaminoethanol |
| Snail mucin | Snail Secretion Filtrate |
| Allantoin | Allantoin |
| Benzoyl peroxide | Benzoyl peroxide |
| Petrolatum | Petroleum jelly |
| Sage | Salvia Officinalis Extract |
| Alpha hydroxy acids | lactic acid, Glycolic Acid |
| Beta hydroxy acids | Salicylic acid |
| Ceramides | Ceramides |
| Hyaluronic acid | Hyaluronic acid |
| D-Panthenol | D-Panthenol |
| Caffeine | Caffeine |
| Bakuchiol | Bakuchiol |
| Chia seed oil | Chia seed oil |
| CBD oil | Cannabidiol |
| Collagen | Collagen |
| Azelaic acid | Azelaic acid |
| Biotin | Biotin |
| Ceramides | Ceramides |
| Hydrocortisone | Hydrocortisone |
| Keratin | Keratin |
| Ferulic acid | Ferulic acid |
| Kojic acid | Kojic acid |
| Hydroquinone | Hydroquinone |
| Glycerol | Glycerin |
| Hemp | Hemp |
| Malic acid | Malic acid |
| Capsaicin | Capsaicin |
| Trehalose | Trehalose |
| Magnesium ascorbyl phosphate | Magnesium ascorbyl phosphate |
| Sodium Ascorbyl Phosphate | Sodium Ascorbyl Phosphate |
| Ascorbic acid glucoside | Ascorbic acid glucoside |
| Arbutin | Arbutin |
| Amino acids and its derivatives | Arginine and Lysine and the like and derivatives |
| | thereof |
| Drugs such as glycyrrhetinic acid, | glycyrrhetinic acid, glycyrrhizic acid and derivatives |
| glycyrrhizic acid and derivatives | thereof, or salts thereof |
| thereof, or salts thereof | |
| Various crude drug extracts | Such as licorice, Chinese quince, Pyrola japonica and |
| | the like |
| Tannin | Tannin |
| Vera Pamil | Vera Pamil |
| Tranexamic Acid | Tranexamic Acid |
| Various Extracts | Such as chestnut rose extract, phellodendron bark |
| | (cork tree bark), coptis rhizome, lithospermum, |
| | Glycyrrhiza glabra, peony, swertia herb, birch, sage, |
| | loquat, carrot, aloe, okra, mallow, iris, grape, coix |
| | seed, rubus suavissimus leaf, sponge gourd, lily, |
| | prunus yedoensis leaf, saffron, cnidium rhizome, |
| | ginger, hypericum, restharrow, garlic, red pepper, |
| | citrus unshiu, Japanese angelica, green tea, seaweed, |
| | yeast, etc. |
| Anti-seborrheic agent | Such as sulfur, thianthl, etc. |
| Anti-inflammatory agent | Such as tranexamic acid, thiotaurine, hypotaurine, etc |
| Antioxidants | Such as dibutyl hydroxy toluene, butyl hydroxy |
| | anisole, and gallic acid esters and the like. |
| Peptides and derivatives thereof | Argireline, Matrixyl (palmitoyl pentapeptide), |
| | Dermaxyl, Pentapeptides, Palmitoyl oligopeptide, |
| | Copper peptides, Signal (or matricins) peptides |
| | Carrier peptides, Neurotransmitter inhibitor peptides |
| Methylxanthines | Theophylline, Caffeine, and Theobromine |

In an aspect the cosmetic compositions can comprise:
0.01 to 10% by weight, preferably from 0.1 to 8% by weight, and more preferably from 0.2 to 5% by weight of one or more skin care active compounds (A),
1.0 to 90% by weight, preferably from 2 to 70% by weight, and more preferably from 5 to 30% by weight of one or more crosslinked polyester components (B),
0 to 90% by weight, preferably from 1 to 70% by weight, and more preferably from 5 to 30% by weight of one or more cosmetically acceptable carriers (C),
0 to 60% by weight of one or more other components commonly used in the cosmetic field (D), based on the total weight of the composition.

In an aspect the cosmetic compositions can comprise:
0.01 to 10% by weight, preferably from 0.1 to 8% by weight, and more preferably from 0.2 to 5% by weight of one or more skin care active compounds (A),
1.0 to 90% by weight, preferably from 2 to 70% by weight, and more preferably from 5 to 30% by weight of one or more crosslinked polyester components (B),
0 to 90% by weight, preferably from 1 to 70% by weight, and more preferably from 5 to 30% by weight of one or more cosmetically acceptable carriers (C),
0 to 60% by weight of one or more other components commonly used in the cosmetic field (D),
based on the total weight of the composition different from A), (B) and (C),
each based on the total weight of the composition.

In an aspect the cosmetic compositions consist of only (A), (B) and optionally (C), and preferably such compositions can consist of:
0.01 to 10% by weight, preferably from 0.1 to 8% by weight, and more preferably from 0.2 to 5% by weight of one or more skin care active compounds (A),
1.0 to 90% by weight, preferably from 2 to 70% by weight, and more preferably from 5 to 30% by weight of one or more crosslinked polyester components (B),
0 to 90% by weight, preferably from 1 to 70% by weight, and more preferably from 5 to 30% by weight of one or more cosmetically acceptable carriers (C),
each based on the total weight of the composition.
   Benefits of Using the Crosslinked Polyester Component (B), in Particular, when Used as a Gel in One or More Solvents or Emollients as Described Herein, in Skin Care Formulations Comprising One or More Skin Care Active Compounds

The compositions of the present invention are useful in particular for stabilizing the skin care active compounds (A) upon storage and their controlled delivery into the skin after application. The stability of the skin care active compounds (A) provided by the composition of the present invention may be measured by any conventional techniques such as high-performance liquid chromatography (HPLC) or visually assessing the color and texture changes of the formulations as described in literature International Journal of Food Properties 2015, 18, 862-879.

The controlled released profile of the skin care active compounds (A) provided by the compositions of the present invention can be established by any conventional method, for example, by ex-Vivo skin penetration techniques through artificial skin using Franz cells as disclosed e.g. in patent U.S. Pat. No. 9,498,409.

In the following some of the benefits of the compositions of the present invention are explained and summarized.

### Skin Nourishment and Conditioning

The compositions of the present invention are useful for regulating the condition of the skin and especially for regulating keratinous tissue condition. Regulation of skin condition, namely mammalian and in particular human skin condition, is often required due to conditions which may be induced or caused by factors internal and/or external to the body. Examples include, environmental damage, radiation exposure (including ultraviolet radiation), chronological aging, menopausal status (e.g., post-menopausal changes in skin), stress, diseases, etc. For instance, “regulating skin condition’ includes prophylactically regulating and/or therapeutically regulating skin condition, and may involve one or more of the following benefits: thickening of skin (i.e., building the epidermis and/or dermis and/or sub-dermal (e.g., subcutaneous fat or muscle) layers of the skin and where applicable the keratinous layers of the nail and hair shaft) to reduce skin atrophy, increasing the convolution of the dermal-epidermal border (also known as the rete ridges), preventing loss of skin elasticity (loss, damage and/or inactivation of functional skin elastin) such as elastosis, sagging, loss of skin recoil from deformation; non-melanin skin discoloration such as under eye circles.

### Sensory Benefits

Oil-based compositions are often oily and tacky in nature and can leave behind an oily residue on skin after application. However, the crosslinked polyester component (B), in particular, when used as a gel in one or more solvents or emollients as described herein, in cosmetic formulations has significantly improved their sensory properties, adding a feel of silky-smooth skin upon application. Due to its thickening effect in formulations, crosslinked polyester component (B) in particular, when used as a gel in one or more solvents or emollients as described herein, adds body and texture to cosmetic composition, providing a cushioning effect. The crosslinked polyester component (B) in particular, when used as a gel in one or more solvents or emollients as described herein, also improves oil absorption, which minimizes the oily feel of cosmetic compositions. In addition, formulations with the crosslinked polyester component (B) in particular, when used as a gel in one or more solvents or emollients as described herein, remain on the skin longer than formulations without the crosslinked polyester component (B) forming long-lasting films that are non-tacky.

### Optical Benefits

In addition to the oily feel of many oil-based formulation, they can often appear shiny or glossy upon application to the skin. However, the crosslinked polyester component (B) in particular, when used as a gel in one or more solvents or emollients as described herein, increases oil absorption in cosmetic applications and therefore decreases gloss of the cosmetic substantially. Gloss values of cosmetic compositions formulated with the crosslinked polyester component (B), in particular, when used as a gel in one or more solvents or emollients as described herein, compared to compositions without it were much lower (measured by the BYK glossmeter). Not only does the crosslinked polyester component (B), in particular, when used as a gel in one or more solvents or emollients as described herein, provide a mattifying effect to the cosmetic formulation, but it also provides a soft-focus effect, blurring lines and improving the optical benefits of the formulation.

### Stabilizing Effects on Skin Care Active Compound

The crosslinked polyester component (B) in particular, when used as a gel in one or more solvents or emollients as described herein, shows a stabilizing effect on various skin care active compounds as described herein, in particular, air sensitive skin care active compounds.

### Effects of Controlled or Modified Release of the Skin Care Active Compound

The crosslinked polyester component (B) in particular, when used as a gel in one or more solvents or emollients as described herein, shows the effect of controlling or modifying the release of the skin care active compounds in cosmetic skin care active comprising compositions. The crosslinked polyester component (B) in particular, when used as a gel in one or more solvents or emollients as described herein, shows in particular the effect of controlling or modifying the transdermal release or diffusion of the skin care active compounds as evidenced by the results of in vitro experiments in a Franz diffusion cell as explained below. The crosslinked polyester component (B) can be used in particular to controlling or modifying the release of the skin care active compounds in cosmetic skin care active comprising compositions, e.g. to sustain, extend or maintain the release at a predetermined rate in order to maintain a constant concentration of the skin care active compounds for a specific period of time, or to enhance or accelerate the penetration of the skin care active compounds, also e.g. in transdermal patches.

### Further Aspects of the Invention

In an aspect the crosslinked polyester component (B) is provided as a gel comprising one or more solvents and/or emollients, preferably selected from a triglyceride solvent, a mono-ester solvent, such as cococaprylate/caprate, and a hydrocarbon solvent to the composition according to the invention.

In an aspect the cosmetic composition according to the invention, which is for the topical application to the skin or mucous membranes.

In an aspect the invention relates thus to the use of at least one crosslinked polyester component (B), comprising a crosslinked polyester which is reaction product of:
(i) at least one compound selected from a polycarboxylic acid, a poly-carboxylic acid ester, and combinations thereof,
(ii) at least one polyol, and
(iii) optionally one or more monofunctional component selected from
   (a) a mono-carboxylic acid; and
   (b) a mono-alcohol,
for the preparation of a cosmetic composition for topical application to the skin or mucous membranes.

The invention accordingly also provides a use of at least one crosslinked polyester component (B), comprising a crosslinked polyester which is reaction product of:
(i) at least one compound selected from a polycarboxylic acid, a poly-carboxylic acid ester, and combinations thereof,
(ii) at least one polyol, and
(iii) optionally one or more monofunctional component selected from
   (a) a mono-carboxylic acid; and
   (b) a mono-alcohol,
in a skin care composition, comprising at least one skin care active compound (A),
as a (controlled) delivery carrier or agent for the skin care active compound (A) to the skin or mucous membranes, and/or
for the (controlled) delivery of skin care active compound (A) to the skin or mucous membranes, and/or
for regulating the condition of the skin and especially for regulating the keratinous tissue condition,
for reducing wrinkle, where the skin care composition is an oil-based cosmetic composition, and/or
for reducing wrinkle, where the skin care composition is an oil-in-water-based cosmetic composition, and/or
for reducing wrinkle, where the skin care composition is a water-in-oil-based cosmetic composition, and/or
for stabilizing the skin care active compound (A) in the cosmetic composition and/or
for thickening the cosmetic composition and/or
for improving the compatibility of skin care active compound (A) with the other ingredients in the cosmetic composition, and/or
for improving the sensory properties of the cosmetic composition, and/or
for improving the optical properties of the cosmetic composition, such as providing a mattifying effect to the formulation, a soft-focus effect, a blurring lines effect, and/or
for improving the water resistance of the cosmetic composition, and/or
for controlling or modifying the release of the skin care active compound (A) in the cosmetic compositions.

In an embodiment the invention also relates to the use of at least one crosslinked polyester component (B) for stabilizing a skin care active compound (A) in the cosmetic composition and/or controlling or modifying the release of a skin care active compound (A) in the cosmetic compositions.

The invention accordingly also provides a method for
stabilizing skin care active compounds (A) in a cosmetic composition, and/or
delivering skin care active compounds (A) in a cosmetic composition, and/or
reducing wrinkles, and/or
stabilizing a cosmetic composition and/or
thickening a cosmetic composition and/or
improving the compatibility of skin care active (A) with other ingredients in a cosmetic composition, and/or
improving the sensory properties of a cosmetic composition, and/or
improving the optical properties of a cosmetic composition, such as providing a mattifying effect to the formulation, a soft-focus effect, a blurring lines effect, and/or
improving the water resistance of a cosmetic composition, controlling the release of the skin care active compounds (A) in a cosmetic composition, which methods comprise the step of adding at least one crosslinked polyester component (B), comprising a crosslinked polyester which is reaction product of:
(i) at least one compound selected from a polycarboxylic acid, a poly-carboxylic acid ester, and combinations thereof,
(ii) at least one polyol, and
(iii) optionally one or more monofunctional component selected from
   (a) a mono-carboxylic acid; and
   (b) a mono-alcohol,
      to said cosmetic composition, comprising at least one skin care active compound (A).

In an embodiment the invention also relates to said method which is for stabilizing a skin care active compound (A) in cosmetic compositions and/or for controlling or modifying the release of the skin care active compound (A) in cosmetic compositions.

### EXAMPLES

The following examples are included to demonstrate various aspects of the present disclosure. It should be appreciated by those of skill in the art that the techniques disclosed in the examples that follow represent techniques discovered by the inventors to function well in the practice of the disclosure, and thus can be considered to constitute preferred modes for its practice. However, those of skill in the art should, in light of the present disclosure, appreciate that many changes can be made in the specific examples which are disclosed and still obtain a like or similar result without departing from the spirit and scope of the disclosure.

### Example 1: Esterification to Prepare Polyester Elastomer Component (B)

In a suitable vessel equipped with agitation, heat, and an ability to distill off water, 130 g dimer acid was added along with 190 g hydrogenated castor oil. Next 50 g squalane was added as solvent. After all ingredients were charged under agitation, the temperature of the mass was raised to 180° C., and water was stripped off as formed. The temperature was held until gelation took place and polymer elastomer was formed. Thereafter the elastomer was broken into a powder by mechanical stirring.

### Example 2: Esterification to Prepare Polyester Elastomer Component (B)

In a suitable vessel equipped with agitation, heat, and an ability to distill off water, 200 g dimer acid was added along with 35 g diglycerol and 30 g oleic acid. Next 30 g squalane was added as solvent. After all ingredients were charged under agitation, the temperature of the mass was raised to 160° C., and water was stripped off as formed. The temperature was held until gelation took place and polymer elastomer was formed. Thereafter the elastomer was broken into a powder by mechanical stirring.

### Example 3: Preparation of Polyester Gel (B) from Polyester Elastomer

After an elastomer powder was produced, such as that made in Example 1, which did not separate upon standing, 32.5 g coco-caprylate/caprate was added to 175 g elastomer. After the elastomer and solvent mixture sit and swell for 1 hour, it was milled with a 3-roll mill, and then diluted with 300 g coco-caprylate/caprate. The milled elastomer solvent mixture was then homogenized in the reactor vessel to produce a creamy, translucent gel of very smooth consistency, suitable for use in personal care formulations.

### Example 4: Esterification to Prepare Polyester Elastomer (B)

Hydrogenated castor oil (141.0 g, 150.08 mmol) was added into a N₂-flushed reactor equipped with a heating mantle, overhead stirrer, reflux condenser, addition funnel, and thermocouple. The stirred content was heated to 100° C. for 1 hour. Titanium (IV) isopropoxide (1.38 g, 3.5 mmol) was added and stirring continued at 100° C. for 5 minutes. To this mixture, a premix of hydrogenated dimer acid (109.1 g, 194.50 mmol) and Di-tert-butyl dicarbonate (DIBOC) (55.2 g, 252.92 mmol) was added and the stirring continued for another 6 hours when the entire reaction mixture turned into a viscous gum. At this point, Cetiol LC (emollient BASF) (125.0 g) and DIBOC (45 g, 103.09 mmol) were added to the mixture and stirring continued at 75° C. for 7 hours, when the entire mixture turned into elastomer powder. Deionized (DI) water (5.0 g) was added and stirred for 1 hour at 75° C. The unreacted water and volatile byproducts were stripped off under low pressure at 75° C. The rheology analysis of the powder sample indicated that the rubber had a modulus (G′) of about 26611 Pa.

### Example 5: Swelling of Polyester Elastomer to Prepare Polyester Gel (B)

25 g of the elastomer powder obtained in synthetic Example 4 and Cetiol LC (75 g) were added into a beaker and stirred at room temperature for 2 hours. The mixture was then subjected to high shear mixing using Silverson's high shear rotor/stator laboratory mixer to produce a creamy, translucent gel of very smooth consistency. The optical microscopic analysis of the gel indicated that it has an average particle size of 20 micron. The rheology analysis of the gel sample indicated a modulus (G′) of about 2540 Pa.

### Example 6: Formulation of a Water-In-Oil Composition with Polyester Gel (B) Comprising Retinol as the Skin Care Active Compound (A)

A water-in-oil composition with the Polyester Gel (B) comprising Retinol with the ingredients shown in the following table was prepared according to the procedure indicated below.

W/O Formulation Containing Retinol

| | | | % W/W | | % W/W |
|---|---|---|---|---|---|
| | | | Polyester | | Si |
| PHASE | INGREDIENT | INCI NAME | Gel | Control | Elastomer |
| A | DM water | Aqua | qs 100 | qs 100 | qs 100 |
| | Disodium EDTA | Disodium EDTA | 0.05 | 0.05 | 0.05 |
| | Easynov | Octyldodecanol (and) Octyldodecyl Xyloside | 5.00 | 5.00 | 5.00 |
| | | (and) PEG-30 Dipolyhydroxystearate | | | |
| | Glycerin | Glycerin | 3.00 | 3.00 | 3.00 |
| B | Euxyl PE 9010 | Phenoxyethanol & Ethylhexyl Glycerin | 1.00 | 1.00 | 1.00 |
| | GTCC | Caprylic/Capric Triglyceride | 10.00 | 10.00 | 10.00 |
| | Skin active | Retinol | 0.50 | 0.50 | 0.50 |
| | Polyester Gel | Polyester Gel (Example 3) | 10.00 | 0.00 | 0.00 |
| | IDD | Isododecane | 0.00 | 10.00 | 0.00 |
| | Si Elastomer | Cyclopentasiloxane (and) Dimethicone | 0.00 | 0.00 | 10.00 |
| | | (and) Dimethicone/Vinyl Dimethicone | | | |
| | | Crosspolymer | | | |

### Procedure:

Combine Phase A ingredients and mix in a vessel until uniform.
Combine Phase B in the main vessel and mix under regular stirring until homogeneous.
Add Phase A to Phase B under mixing, until uniform.

### Active Release Study Using Formulation Example 6

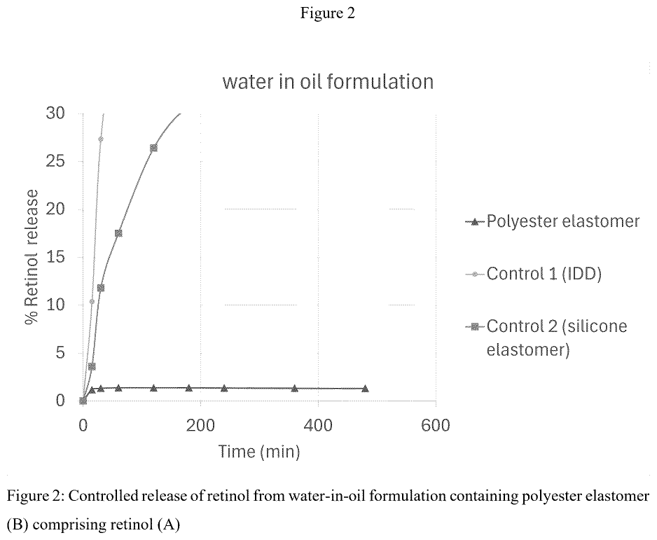

Typically, 300-400 mg of formulation were spread over a shiny side of the Strat-M® membrane for Transdermal Diffusion Testing. The membrane was placed in a Franz diffusion cell in such a way that the side on which formulation was applied faces upwards while the other side of the membrane is in direct contact with the receiver medium, 1-octanol. The release rate experiment was carried out at 25±1° C. Samples (−0.5 mL) were withdrawn from the receiver medium at predetermined time intervals 15, 30, 60, 120, 180, 240, 360, & 480 mins, and the volume sampled was replaced with fresh receptor medium (1-octanol). Sink condition was achieved by selecting a receiver medium with a high capacity to dissolve the retinol. Typically, the receiver medium and amount of sample applied was decided so that the active concentration does not exceed 10-20% of the active solubility in receptor medium at the end of the release test. The % retinol release was plotted against the time. Retinol was quantified using reversed phase HPLC (C18 column). Retinol release was monitored with UV detector at 300 nm using Acetonitrile-Water-Isopropyl alcohol (IPA) mobile phase. As represented in FIG. 2, upon contact with 1-octanol, it was found that the composites release retinol in a controlled manner with only 1 wt % of total retinol get released in first one hour and maintained throughout the study.

As further shown in FIG. 2, the formulation containing polyester elastomer (B) provided controlled release of retinol (component (A)) (about 1 wt %) throughout the course of the study. However, in control 1 (replacing polyester gel (B) by isododecane) and control 2 (replacing polyester elastomer (B) by a silicon elastomer), the release of retinol was uncontrolled and increased with time.

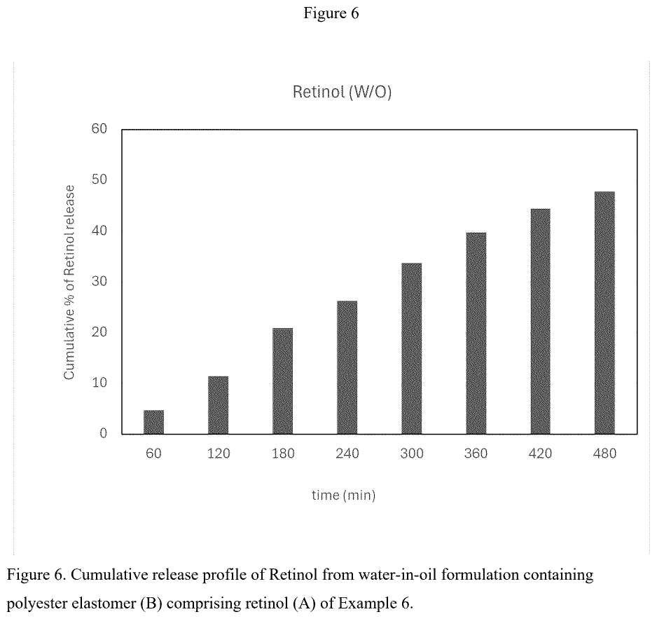

FIG. 6 shows accordingly the almost linear cumulative release profile of retinol from the water-in-oil formulation containing polyester elastomer (B) comprising retinol (A) of Example 6.

### Example 7: Formulation of a Water-In-Oil Composition with Polyester Gel (B) Comprising Niacinamide as the Skin Care Active Compound (A)

A water-in-oil composition containing Niacinamide as shown in the following table was prepared by the procedure indicated below.

W/O Formulation Containing Niacinamide

| | | | % W/W | | % W/W |
|---|---|---|---|---|---|
| | | | Polyester | | Si |
| PHASE | INGREDIENT | INCI NAME | Gel | Control | Elastomer |
| A | DM water | Aqua | qs 100 | qs 100 | qs 100 |
| | Disodium | Disodium EDTA | 0.05 | 0.05 | 0.05 |
| | EDTA | | | | |
| | Skin active | Niacinamide | 0.50 | 0.50 | 0.50 |
| | Easynov | Octyldodecanol (and) Octyldodecyl | 5.00 | 5.00 | 5.00 |
| | | Xyloside (and) PEG-30 | | | |
| | | Dipolyhydroxystearate | | | |
| | Gycerin | Glycerin | 3.00 | 3.00 | 3.00 |
| B | Euxyl PE9010 | Phenoxyethanol & Ethylhexyl Glycerin | 1.00 | 1.00 | 1.00 |
| | GTCC | Caprylic/Capric Triglyceride | 10.00 | 10.00 | 10.00 |
| | Si Elastomer | Cyclopentasiloxane (and) Dimethicone | 0.00 | 0.00 | 10.00 |
| | | (and) Dimethicone/Vinyl Dimethicone | | | |
| | | Crosspolymer | | | |
| | Polyester Gel | Polyester Gel (Example 3) | 10.00 | 0.00 | 0.00 |
| | IDD | Isododecane | 0.00 | 10.00 | 0.00 |

### Procedure:

Combine Phase A ingredients and mix in a vessel until uniform.
Combine Phase B in the main vessel and mix under regular stirring until homogeneous.
Add Phase A to Phase B under mixing, until uniform.

### Active Release Study Using Formulation Example 7

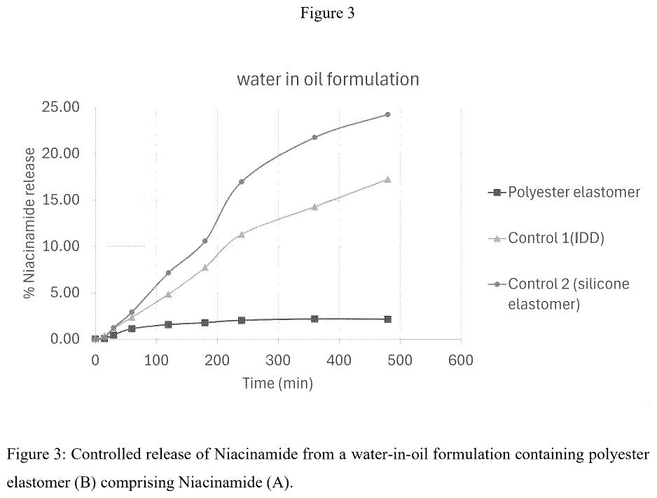

Typically, 300-400 mg of formulation were spread over a shiny side of the Strat-MR membrane for Transdermal Diffusion Testing. The membrane was placed in a Franz diffusion cell in such a way that the side on which formulation was applied faces upwards while the other side of the membrane is in direct contact with the receiver medium, 1-octanol. The release rate experiment was carried out at 25±1° C. Samples (0.5 mL) were withdrawn from the receiver medium at predetermined time intervals 15, 30, 60, 120, 180, 240, 360, & 480 mins, and the volume sampled was replaced with fresh receptor medium (1-octanol). The % niacinamide release was plotted against the time. Niacinamide was quantified using reversed phase HPLC (C18 column). Niacinamide release was monitored with UV detector at 300 nm using Acetonitrile-Water-Isopropyl alcohol (IPA) mobile phase. As represented in FIG. 3, upon contact with 1-octanol, it was found that the composites release niacinamide in a controlled manner where only 1 wt % of total niacinamide get released in first 5 hours and maintained throughout the study.

As further shown in FIG. 3, the formulation containing polyester elastomer (B) provided a controlled release of niacinamide (A) (about 2 wt %) throughout the course of the study. However, in control 1 (replacing polyester gel (B) by isododecane) and control 2 (replacing polyester elastomer (B) by a silicon elastomer), the release of retinol was uncontrolled and increased with time.

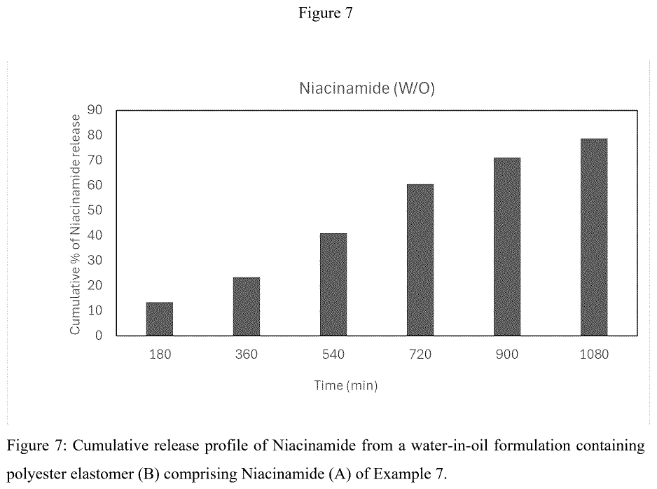

FIG. 7 accordingly shows an almost linear cumulative release profile of Niacinamide from the water-in-oil formulation containing polyester elastomer (B) comprising Niacinamide (A) of Example 7.

### Example 8: Formulation of a Oil-In-Water Composition with Polyester Gel (B) Comprising Niacinamide as the Skin Care Active Compound (A)

An oil-in-water composition with the Polyester Gel (B) comprising Niacinamide with the ingredients shown in the following table was prepared according to the procedure indicated below.

O/W Formulation Containing Niacinamide

| | | | % W/W | | % W/W |
|---|---|---|---|---|---|
| | | | Polyester | | Si |
| PHASE | INGREDIENT | INCI NAME | Gel | Control | Elastomer |
| A | DM water | Aqua | qs 100 | qs 100 | qs 100 |
| | Disodium EDTA | Disodium EDTA | 0.05 | 0.05 | 0.05 |
| | Skin active | Niacinamide | 0.5 | 0.5 | 0.5 |
| | Seigel 305 | Polyacrylamide & C13-14 Isoparaffin | 2.50 | 2.50 | 2.50 |
| | | & Laureth-7 | | | |
| | Glycerin | Glycerin | 3.00 | 3.00 | 3.00 |
| B | Euxyl PE9010 | Phenoxyethanol & Ethylhexyl | 1.00 | 1.00 | 1.00 |
| | | Glycerin | | | |
| | GTCC | Caprylic/Capric Triglyceride | 10.00 | 10.00 | 10.00 |
| | Polyester Gel | Polyester Gel (Example 3) | 10.00 | 0.00 | 0.00 |
| | IDD | Isododecane | 0.00 | 10.00 | 0.00 |
| | Si Elastomer | Cyclopentasiloxane (and) | 0.00 | 0.00 | 10.00 |
| | | Dimethicone (and) | | | |
| | | Dimethicone/Vinyl Dimethicone | | | |
| | | Crosspolymer | | | |

### Procedure:

Combine Phase A ingredients in the main vessel and mix under regular stirring until homogeneous.
Combine Phase B ingredients and mix in a vessel until uniform.
Add Phase B to the main kettle which has phase A under mixing, until uniform.

### Active Release Study Using Formulation Example 8

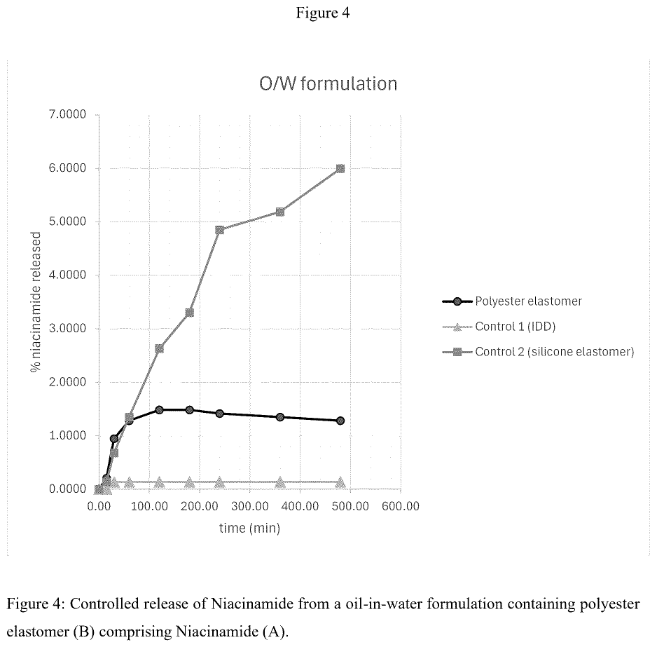

Typically, 300-400 mg of formulation were spread over a shiny side of the Strat-M® membrane for Transdermal Diffusion Testing. The membrane was placed in a Franz diffusion cell in such a way that the side on which formulation was applied faces upwards while the other side of the membrane is in direct contact with the receiver medium, 1-octanol. The release rate experiment was carried out at 25±1° C. Samples (0.5 mL) were withdrawn from the receiver medium at predetermined time intervals 15, 30, 60, 120, 180, 240, 360, & 480 mins, and the volume sampled was replaced with fresh receptor medium (1-octanol). The % niacinamide release was plotted against the time. Niacinamide was quantified using reversed phase HPLC (C18 column). Niacinamide release was monitored with UV detector at 300 nm using Acetonitrile-Water-Isopropyl alcohol (IPA) mobile phase. As represented in FIG. 4, upon contact with 1-octanol, it was found that the composites release niacinamide in a controlled manner where only 1 wt % of total niacinamide get released in first 5 hours and maintained throughout the study.

As further shown in FIG. 4, the formulation containing polyester elastomer (B) provided a controlled release of niacinamide (A) (about 1.5 wt %) throughout the course of the study. However, in control 2 (replacing polyester elastomer (B) by a silicon elastomer) the release was uncontrolled and increased with time. In control 1 (replacing polyester gel (B) by isododecane), there was no significant release of active over time.

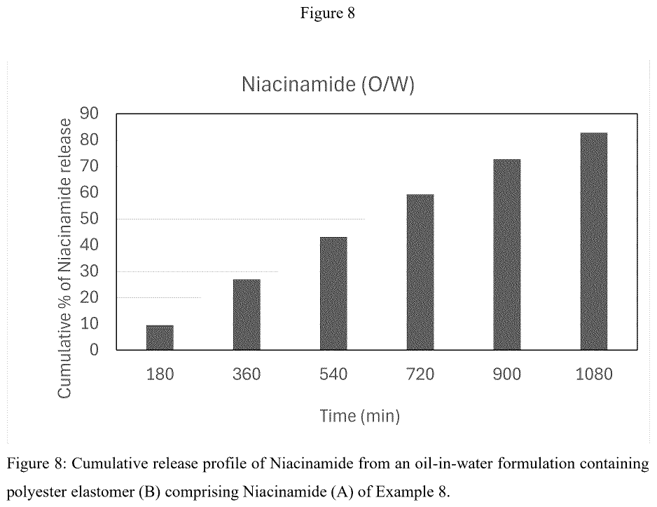

FIG. 8 accordingly shows an almost linear cumulative release profile of Niacinamide from the oil-in-water formulation containing polyester elastomer (B) comprising Niacinamide (A) of Example 8.

### Example 9: Formulation with Different Loading of Polyester Gel (B) and Actives (Retinol and Niacinamide) as the Skin Care Active Compound (A)

Formulation Containing Different Concentration of Polyester Gel

| | | | % W/W | % W/W | % W/W | % W/W |
|---|---|---|---|---|---|---|
| | | | Polyester | Polyester | Polyester | Polyester |
| | | | gel | gel | gel | gel |
| PHASE | INGREDIENT | INCI NAME | 5 wt % | 10 wt % | 15 wt % | 75 wt % |
| A | DM water | Aqua | qs 100 | qs 100 | qs 100 | qs 100 |
| | Disodium | Disodium EDTA | 0.05 | 0.05 | 0.05 | 0.05 |
| | EDTA | | | | | |
| | Skin active | Niacinamide | 0.5 | 0.5 | 0.5 | 0.5 |
| | Easynov | Octyldodecanol and | 0 | 0 | 0 | 4 |
| | | Octyldodecyl Xyloside | | | | |
| | | and PEG-30 | | | | |
| | | Dipolyhydroxystearate | | | | |
| | Seigel 305 | Polyacrylamide & | 2.5 | 2.5 | 2.5 | 0 |
| | | C13-14 Isoparaffin & | | | | |
| | | Laureth-7 | | | | |
| | Gycerine | Glycerin | 3 | 3 | 3 | 0 |
| B | Euxy1 | Phenoxyethanol & | 1 | 1 | 1 | 1 |
| | PE9010 | Ethylhexyl Glycerin | | | | |
| | GTCC | Caprylic/Capric | 15 | 10 | 5 | 10 |
| | | Triglyceride | | | | |
| | Polyester Gel | Polyester Gel | 5 | 10 | 15 | 75 |
| | | (Example 3) | | | | |
| | Skin active | Retinol | 0 | 0 | 0 | 0.5 |

### Procedure:

Combine Phase A ingredients in the main vessel and mix under regular stirring until homogeneous.
Combine Phase B ingredients and mix in a vessel until uniform.
For formulation containing 5, 10 and 15 wt % polyester elastomer, add Phase B to the main kettle which has phase A under mixing, until uniform.
For formulation containing 75 wt % polyester elastomer, add Phase A to the main kettle which has phase B under mixing, until uniform.

### Measurement of the Cumulative Release Profiles of Actives from the Polyester Elastomer Formulations of Example 6, 7, 8 and 9

Analyzing the cumulative release profile of an active over multiple cycles is a technique used to determine the efficacy and consistency of an active delivery system. This is especially relevant for controlled-release formulations, which are designed to release an active in a control manner over a prolonged period. The process involves measuring the cumulative amount of active released at a specific time point over two or more “cycles” and analyzing the resulting data. Thus, 300-400 mg of the formulation were spread over a shiny side of the Strat-M® membrane for Transdermal Diffusion Testing. The membrane was placed in a Franz diffusion cell in such a way that the side on which formulation was applied faces upwards while the other side of the membrane is in direct contact with the receiver medium, 1-octanol. The release rate experiment was carried out at 25±1° C. Samples (0.5 mL) were withdrawn from the receiver medium at a fixed time interval of 60 mins (retinol) and 180 mins (niacinamide). After each sampling, replaced the entire volume of the receptor medium with fresh sample to maintain “sink conditions”. The procedure was repeated for multiple cycles. The cumulative amount of active released at each cycle was the sum of the active in the current sample plus the active from all previous samples. To normalize the data, the cumulative amount was expressed as a percentage of the total active loaded into the system. The cumulative release profiles for multiple cycles are very similar, it indicates that the active delivery system is stable, and the release behavior is highly reproducible.

As shown in FIGS. 6, 7 and 8, the active delivery system comprising polyester elastomers can consistently deliver a predictable amount of active over an extended time period.

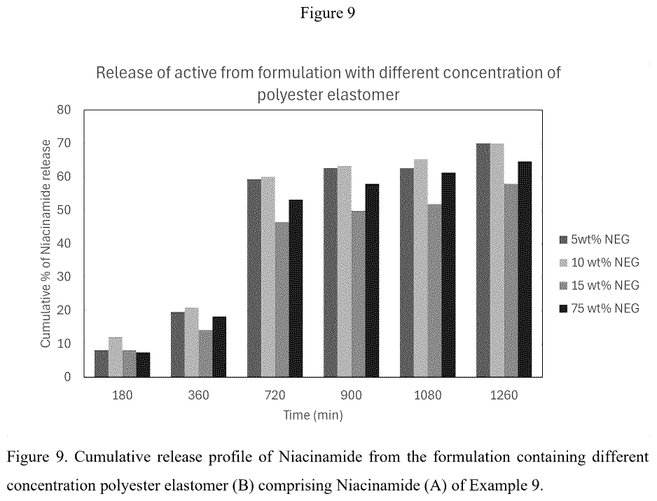

The influence of polyester elastomer loading on the cumulative release profile of the active compound was evaluated using the model formulations containing 5, 10, 15, and 75 wt % elastomer of Example 9. As illustrated in FIG. 9, the release profiles remained largely consistent across the different elastomer loadings, indicating minimal impact on the release kinetics. This consistency underscores the robustness of the delivery system, demonstrating its ability to maintain a stable release profile over a broad range of elastomer concentrations.

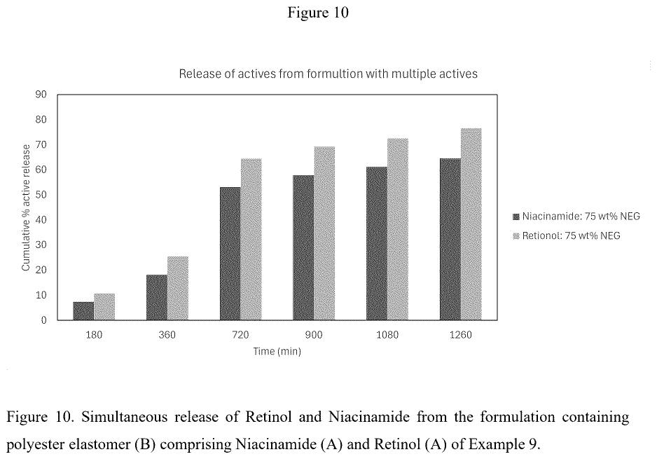

Further, simultaneous active release in multi-active cosmetic formulations is crucial for achieving enhanced efficacy and stability while providing a simplified and targeted consumer experience. This approach addresses the significant challenge of combining multiple incompatible ingredients and ensuring their optimal function on the skin. As illustrated in FIG. 10, the release profiles of both retinol and niacinamide in the formulation containing 75 wt % of the polyester gel remained largely consistent when they are incorporated into a single formulation.

### Example 10: Formulation of a Water-In-Oil Composition with Polyester Gel (B) Comprising Vitamin C (A)

A water-in-oil composition with the Polyester Gel (B) comprising Vitamin C with the ingredients shown in the following table was prepared according to the procedure indicated below

F1-Formulation
Polyester Gel Comprising Vitamin C

| Phase | INCI Name | Wt (%) |
|---|---|---|
| A | Polyester Gel (example 3) | 60 |
| | Vitamin C | 10 |
| | Glycerin | 30 |

C1-Formulation without Polyester Gel Comprising Vitamin C

| Phase | INCI Name | Wt (%) |
|---|---|---|
| A | Aqua | 59 |
| | Ammonium Acryloyldimethyltaurate/VPCopolymer | 1 |
| | Vitamin C | 10 |
| | Glycerin | 30 |

### Procedure for Formula F1:

Mix Ascorbic acid with Glycerin and mix until it dissolves.
Then add this mixture slowly to the Polyester gel and mix until homogenous Procedure for C1:
Mix Ascorbic acid in water and mix until it dissolves.
Then add glycerin and slowly disperse the polymer, Aristoflex AVC to make a hydrogel
Mix until homogenous

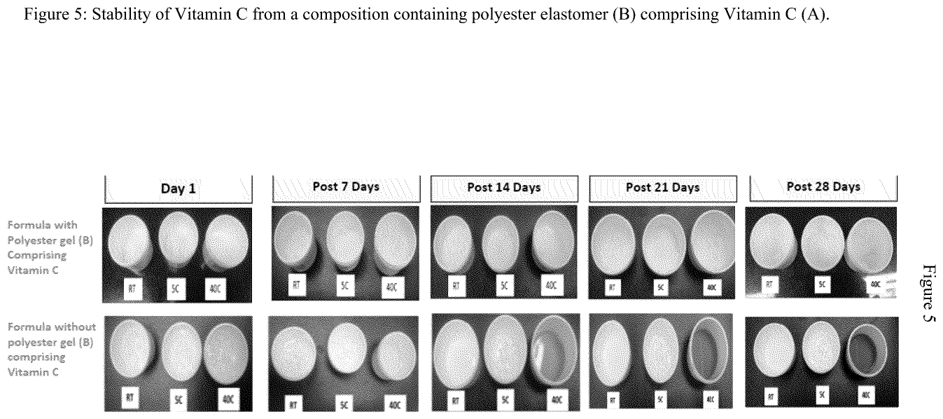

FIG. 5 shows the results of accelerated thermal stability observations:
The formulations were kept for accelerated thermal stability at 5° C., RT (room temperature), 40° C.
The formulations were checked for the following parameters at different time intervals (day 1, 7, 14 21 and 28):
   a) Physical Impact:
      i. Color
      ii. Oil bleeding/Syneresis
      iii. Consistency.

As shown in FIG. 5, the formulation without polyester elastomer (B) underwent faster color change indicating a faster degradation of vitamin C compared to the formulation containing polyester elastomer (B).

1. A composition comprising:
(A) at least one skin care active compound, and
(B) at least one crosslinked polyester component, wherein the at least one crosslinked polyester component comprises a crosslinked polyester which is a reaction product of:
(i) at least one compound selected from the group consisting of poly-carboxylic acids, poly-carboxylic acid esters, and combinations thereof, and
(ii) at least one polyol.

2. The composition of claim 1,
wherein the crosslinked polyester of (B) which is further the reaction product of:
(iii) at least one mono-carboxylic acid; or
(iii) at least one mono-carboxylic acid; and (iv) one or more mono-alcohol.

3. The composition claim 1, which is suitable for use as a cosmetic composition.

4. The composition of claim 2, comprising
(C) at least one cosmetically acceptable carrier different from components (A) and (B); or
(C) at least one cosmetically acceptable carrier and (D) one or more other components commonly used in the cosmetic field, wherein (C) is different from components (A), (B), and (D), and (D) is different from components (A), (B), and (C).

5. The composition of claim 1, wherein the at least one skin care active compound (A) is selected from the group consisting of ascorbyl palmitate, kojic acid, lucinol, and oil-soluble licorice extract, wrinkle preventers, circulation improving ingredients, antimicrobial ingredients, vitamins, and combinations thereof.

6. The composition of claim 4, wherein the at least one cosmetically acceptable carrier (C) is selected from the group consisting of water, solvents, emollients, fatty acids, fatty alcohols, film formers, emulsifiers, thickeners, and combinations thereof.

7. The composition of claim 1, wherein:
the at least one compound (i) is selected from the group consisting of
(1) one or more poly-carboxylic acids of formula (I) 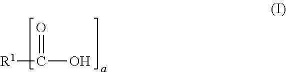
wherein
R¹ is selected from the group consisting of C2-C52 alkyl group, C2-C52 heteroalkyl group, C2-C52 alkene group, C2-C52 heteroalkene group, C3-C52 cyclic group, and C2-C52 heterocyclic group, and
a is an integer from 2 to 10, or
(2) one or more carboxylic acid esters of formula (II) 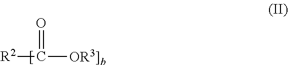
wherein
R² is C1-C22 alkyl group, C2-C22 alkylene group, or C3-C22 cyclic group;
R³ is C2-C200 alkyl group, C2-C200 heteroalkyl group, C2-C200 alkene group, C2-C200 heteroalkene group, C2-C200 alkyne group, C2-C200 heteroalkyne group, C2-C200 cyclic group, or C2-C200 heterocyclic group; and
b is an integer from 2 to 10, and
(3) a combination thereof, and
the at least one polyol (ii) is a polyol of formula (III) 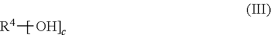
wherein
R⁴ is C2-C200 alkyl group, C2-C200 heteroalkyl group, C2-C200 alkene group, C2-C200 heteroalkene group, C2-C200 alkyne group, C2-C200 heteroalkyne group, C3-C200 cyclic group, or C2-C200 heterocyclic group; and
c is an integer from 2 to 10

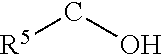

8. The composition of claim 1, wherein:
the at least one compound (i) is one or more poly-carboxylic acids selected from the group consisting of di-carboxylic acids, tri-carboxylic acids, and combinations thereof;
the at least one polyol (ii) is selected from the group consisting of diols, triols, and combinations thereof.

9. (canceled)

10. The composition of claim 1, wherein the at least one crosslinked polyester component (B) has a gel fraction which is greater than 20%.

11. The composition of claim 1, wherein the at least one crosslinked polyester component (B) has a swelling ratio of from about 1 gram/gram to about 15 gram/gram.

12. A The composition of claim 1, wherein the at least one crosslinked polyester component (B) is comprised of particles of a size from about 1 μm to about 500 μm as measured by a laser diffraction particle size analyzer.

13. The composition of claim 1, wherein the at least one crosslinked polyester component (B) is obtained by:
(a) reacting the at least one compound (i) and the at least one polyol (ii)
to obtain the crosslinked polyester component (B); and
(b) subjecting the crosslinked polyester of (B) obtained in (a) to shear force to obtain the crosslinked polyester component (B) as a gel;
or
(c) adding one or more solvents, one or more emollients, or a combination thereof to the crosslinked polyester component (B) obtained in (a), allowing the resulting mixture to swell in one or more solvents, one or more emollients, or combination thereof and subjecting the swollen mixture to shear force to obtain the at least one cross-linked polyester component (B) as a gel.

14. (canceled)

15. The composition of claim 13, wherein the shear force in (b) and (c) is carried out with a batch mixer, planetary mixer, single or multiple screw extruder, dynamic or static mixer, colloid mill, homogenizer, sonolator, three roll mill, or a combination thereof.

16. (canceled)

17. The composition of claim 13, wherein the amount of the one or more solvents, one or more emollients, or a combination thereof added in (c) is such that the resulting crosslinked polyester component (B) as a gel comprises from about 10% to about 95%, weight by weight of the one or more solvents, one or more emollients, or a combination thereof based on the total weight of the resulting composition.

18. (canceled)

19. (canceled)

20. The composition of claim 3, wherein the one or more other components commonly used in the cosmetic field (D) are selected from the group consisting of pigment, humectant, vitamin, moisturizer, conditioner, oil, suspending agent, surfactant, emulsifier, preservative, rheology modifier, pH adjustor, reducing agent, anti-oxidant, foaming agents, de-foaming agents, chelating agents, gums or thickeners, oils, waxes, a fragrances, essential oils, and combinations thereof.

21. The composition of claim 3, comprising:
0.1 to 85% by weight, of at least one skin care active compound (A),
1.0 to 80% by weight of at least one crosslinked polyester component (B),
0 to 90% by weight of at least one cosmetically acceptable carrier (C),
0 to 60% by weight of one or more other components commonly used in the cosmetic field (D),
based on the total weight of the composition.

22. (canceled)

23. A personal care formulation comprising the composition of claim 1, wherein the personal care formulation is selected from the group consisting of skin creams, facial creams, hair care products, protective creams, color cosmetics, foundations, blushes, makeup, and mascara.

24. A composition claims, consisting of:
(A) at least one skin care active compound, and
(B) at least one crosslinked polyester component, wherein the at least one crosslinked polyester component comprises a crosslinked polyester which is a reaction product of:
(i) at least one compound selected from the group consisting of poly-carboxylic acids, poly-carboxylic acid esters, and combinations thereof, and
(ii) at least one polyol; or
(A) at least one skin care active compound;
(B) at least one crosslinked polyester component, wherein the at least one crosslinked polyester component comprises a crosslinked polyester which is a reaction product of:
(i) at least one compound selected from the group consisting of poly-carboxylic acids, poly-carboxylic acid esters, and combinations thereof, and
(ii) at least one polyol; and
(C) at least one cosmetically acceptable carrier.

25.-28. (canceled)

29. A personal care formulation comprising:
(A) at least one skin care active compound, and
(B) at least one crosslinked polyester component, wherein the at least one crosslinked polyester component comprises a crosslinked polyester which is a reaction product of:
(i) at least one compound selected from the group consisting of poly-carboxylic acids, poly-carboxylic acid esters, and combinations thereof, and
(ii) at least one polyol;
wherein the personal care formulation comprising the at least one crosslinked polymer component (B) provides a benefit compared to a personal care formulation without the at least one crosslinked polymer component (B), wherein the benefit is selected from the group consisting of:
an increase in stability of the at least one skin care active compound (A);
improved delivery of the at least one skin care active compound (A);
a reduction in wrinkles;
an increase in stability of the personal care formulation;
an increase in thickness of the personal care formulation;
improved compatibility of the at least one skin care active compound (A) with other ingredients in the personal care formulation;
improved sensory properties of the personal care formulation;
improved optical properties of the personal care formulation;
improved water resistance of the personal care formulation;
controlled release of the at least one skin care active compound (A) in the personal care formulation; and combinations thereof.

30. (canceled)

31. The composition of claim 2, wherein:
the at least one mono-carboxylic acid (iii) is a mono-carboxylic acid of formula (IV) 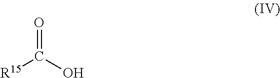
wherein
R⁵ is C2-C52 alkyl group, C2-C52 heteroalkyl group, C2-C52 alkene group, C2-C52 heteroalkene group, C3-C52 cyclic group, or C2-C52 heterocyclic group; and
the one or more mono-alcohol (iv) is a mono-alcohol of formula (V) 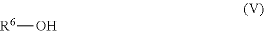
wherein
R⁶ is C2-C52 alkyl group, C2-C52 heteroalkyl group, C2-C52 alkene group, C2-C52 heteroalkene group, C3-C52 cyclic group, or C2-C52 heterocyclic group.
